(19) United States
(12) Patent Application Publication
(10) Pub. No.: US 2015/0056697 A1
(43) Pub. Date: Feb. 26, 2015

(54) PLURIPOTENT STEM CELLS AND METHOD OF STIMULATING AND EXTRACTING NON-EMBRYONIC PLURIPOTENT STEM CELLS FROM MAMMAL BLOOD AND USING RECONSTITUTED PLURIPOTENT STEM CELLS TO TREAT DISEASES INCLUDING CHRONIC OBSTRUCTIVE PULMONARY DISEASE

(71) Applicant: Royal Medical Group, PLLC, Henderson, NV (US)

(72) Inventors: DANIEL F. ROYAL, henderson, NV (US); Henry E. Young, Macon, GA (US); Seth Dyal, Henderson, NV (US)

(21) Appl. No.: 14/530,253

(22) Filed: Oct. 31, 2014

Related U.S. Application Data
(62) Division of application No. 13/362,993, filed on Jan. 31, 2012.
(60) Provisional application No. 61/437,705, filed on Jan. 31, 2011.

(51) Int. Cl. C12N 5/074

(52) U.S. Cl. CPC C12N 5/0607
USPC 435/325

(57) ABSTRACT

Stimulating tissue resident pluripotent stem cells in a manner that the respective subject (e.g., human) acts as its own sterile bioreactor for in vivo stem cell proliferation thus eliminating the need to isolate, cultivate, maintain, proliferate and release stem cells ex vivo. The stimulation mobilizes excess pluripotent stem cells into the peripheral vasculature where the pluripotent stem cells can either migrate to damaged tissues and/or be harvested by simple venipuncture, thus eliminating potential morbidity and mortality elicited from harvesting tissue from solid tissue sites. The pluripotent stem cells are separated from the blood by gravity sedimentation, after which the pluripotent stem cells can easily be aspirated from the white blood cells and red blood cells. Billions of pluripotent stem cells can be generated in this fashion for infusion/injection into the body, via the vasculature, and into the organ(s) in need of tissue repair and regeneration.

### CROSS REFERENCE

This application is a divisional of U.S. patent application Ser. No. 13/362,993 filed on Jan. 31, 2012, which claims the benefit of U.S. Provisional Patent Application No. 61/437,705 filed on Jan. 31, 2011, both of which are incorporated herein by reference in their entirety for all purposes.

### FIELD OF THE INVENTION

The embodiments of the present invention relate to a method of expanding the number of non-embryonic, pluripotent stem cells and their use for the treatment of diseases, such as chronic obstructive pulmonary disease (COPD), muscular dystrophy, general neuropathies, diabetic neuropathies, Hypotonia, ALS and autoimmune diseases.

### BACKGROUND

The use of embryonic stem cells has faced and continues to face moral challenges from many governments, doctors and other interested parties. Thus, the use of non-embryonic stem cells has become a primary focus of researchers in the stem cell space. One problem with non-embryonic stem cells has been isolating and expanding their numbers in human (or animal) tissue.

Accordingly, there is a need for expanding the numbers of non-embryonic stem cells available in human tissue and developing methods to harvest, reconstitute and re-introduce the non-embryonic stem cells into subjects for use in treating COPD and other diseases.

### SUMMARY

The embodiments of the present invention relate to method of expanding the number of non-embryonic, pluripotent stem cells and their use for the treatment of incurable diseases. In one embodiment, a method comprises broadly: (i) utilizing a stem cell stimulant to increase the number of non-embryonic, pluripotent stem cells in the tissue and/or bloodstream of a subject; (ii) drawing blood from the subject; (iii) separating the non-embryonic, pluripotent stem cells from other blood constituents; (iv) re-constituting the non-embryonic, pluripotent stem cells; and (v) infusing or returning the re-constituted, non-embryonic, pluripotent stem cells into the subject to treat an identified disease.

The embodiments of the present invention are directed to in vivo multiplying pluripotent stem cells located in the connective tissue niches throughout the bodies of mammals, including humans. In one embodiment, the in vivo multiplied pluripotent stems cells are mobilized to the peripheral vasculature of the body. In one embodiment, the in vivo pluripotent stem cells are harvested from the peripheral blood circulation via venipuncture. In one embodiment, hematopoietic elements are liberated from pluripotent stem cells by gravity sedimentation at zero to 10 degrees centigrade for 24 to 72 hours. In one embodiment, the pluripotent stem cells are infused back into the vasculature as a bolus of pluripotent stem cells by intravenous (IV) infusion. In one embodiment, the pluripotent stem cells are nebulized into the lung airways to the alveolar sacs to heal cells lining the lung from bronchi to the avelor sacs. Other infusion methods are useful as well.

Stem cell propagation ex vivo involves stem cells grown in culture which are routinely supplemented with animal and/or human serum to optimize and enhance cell viability. The constituents of serum include water, amino acids, glucose, albumins, immunoglobulins and one or more bioactive agents. Potential bioactive agents present in serum include agents that induce proliferation, agents that accelerate phenotypic expression, agents that induce differentiation, agents that inhibit proliferation, agents that inhibit phenotypic expression and agents that inhibit differentiation. Unfortunately, the identity(ies), concentration(s), and potential combinations of specific bioactive agents contained in different lots of serum is/are unknown. One or more of these unknown agents in serum have shown a negative impact on the isolation, cultivation, cryopreservation and purification of lineage-uncommitted blastomere-like stem cells. Similarly, where feeder layers for stem cells were employed, contamination of stem cell cultures with feeder layer specific components, and especially viruses, frequently occurs.

Alternatively, serum-free media are known for general cell culture, and selected pluripotent stem cells have been propagated in such medium containing a plurality of growth factors as described in United States Publication Application Nos. 2005/0164380 and 2003/0073234; U.S. Pat. Nos. 6,617,159 and 6,117,675; and European Patent No. 1,298,202.

Previously, pluripotent stem cells of human and mammalian origin have been isolated from bone marrow aspirates, adipose tissue, and connective tissue in general. The steps required for extraction of pluripotent stem cells from these tissues is difficult and time consuming, with multiple chances for contamination of the cultures.

Other variations, embodiments and features of the present invention will become evident from the following detailed description, drawings and claims.

### BRIEF DESCRIPTION OF THE DRAWINGS

FIG. 1 illustrates a flow chart detailing a first procedure according to the embodiments of the present invention;

FIG. 2 illustrates a flow chart detailing a second procedure according to the embodiments of the present invention;

FIGS. 3a-3l illustrate pre-treatment patient questionnaires and corresponding post-treatment questionnaires of Parkinson's patients being treated according to the embodiments of the present invention;

FIGS. 4a-4d illustrates pre-treatment patient and post-treatment questionnaires of COPD patients according to the embodiments of the present invention; and

FIGS. 5a-5b illustrates pre-treatment patient and post-treatment questionnaires of a MS patient according to the embodiments of the present invention.

### DETAILED DESCRIPTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

The embodiments of the present invention involve a method of expanding the number of non-embryonic, pluripotent stem cells and their use for the treatment of diseases, many which are incurable. While numerous diseases are suitable for treatment using the method according to the embodiments of the present invention, the detailed description below focuses on COPD. Those skilled in the art will recognize that COPD is only an exemplary disease treatable via the method according to the embodiments of the present invention.

COPD is a lung disease that makes it hard to breathe. COPD is caused by damage to the lungs over many years, usually from smoking, but also non-smoking factors such as biomass fuels, occupational exposure to dusts and gasses, history of pulmonary tuberculosis, respiratory tract infections during childhood, indoor and outdoor pollutants, poor socioeconomic status and asthma. In one large U.S. Study (Barnes, 2009), poorly controlled asthma was found to be a risk even greater than tobacco smoking Over time, breathing tobacco smoke and other pollutants, irritates the airways and destroys the stretchy fibers in the lungs. Secondhand smoke is also bad.

COPD is often a mix of two diseases: 1) Chronic Bronchitis, in which the airways that carry air to the lungs become inflamed and generate an overabundance of mucus which can narrow or block the airways, making it hard to breathe and 2) emphysema, in which the tiny air sacs in the lungs become like balloons. As one breathes in and out, the air sacs get bigger and smaller to move air through the lungs. But with emphysema, these air sacs are damaged and lose their stretchability allowing less air to get in and out of the lungs, which makes one feel short of breath.

COPD gets worse over time and lung damage cannot be reversed. It usually takes many years for the lung damage to start causing symptoms, so COPD is most common in people who are older than 60 years of age.

The main symptoms of COPD are: a long-lasting (chronic) cough, mucus that comes up when one coughs and shortness of breath that gets worse upon exertion. As COPD gets worse, one may be short of breath even when one does simple things like getting dressed or fixing a meal. It gets harder to eat or exercise, and breathing takes much more energy. People often lose weight and get weaker.

At times, one's symptoms may suddenly flare up and get much worse. This is called a COPD exacerbation. An exacerbation can range from mild to life-threatening. The longer you have COPD, the more severe these flare-ups will be.

For smokers, the only way to slow down COPD is to quit smoking. This is the most important thing one can do. No matter how long one has smoked or how serious one's COPD is, quitting smoking can help stop the damage to one's lungs. Another method is to remove oneself from environmental pollutants and irritants as much as possible. Yet another is to participate in pulmonary rehabilitation. A doctor can prescribe this for patients with COPD.

Pulmonary rehabilitation is an important therapy in the management of patients with symptomatic COPD, because it improves the perception of dyspnea, exercise tolerance and health-related quality of life. The effectiveness of pulmonary rehabilitation has been evaluated using many different outcome tools. Functional dyspnea improvement has been documented using the Medical Research Council (MRC) scale and the baseline and transitional dyspnea index (BDI/TDI), whereas exercise dyspnea has been shown to improve using the visual analog scale (VAS) and the Borg scale. Increased exercise tolerance has been most frequently documented using the 6-min walk distance (6MWD). Health-related quality of life has been evaluated with disease-specific tools (e.g., the St. George's Respiratory Questionnaire (SGRQ)) and the Chronic Respiratory Disease Questionnaire (CRQ) and also with more generic questionnaires, such as the Short Form-36 (SF-36). Although all of the aforementioned tools are useful, they are time consuming and require training to be used and interpreted correctly. The health-care practitioner could be helped by well-validated information providing a guide to help select the simplest tools that adequately capture the changes induced by pulmonary rehabilitation.

Doctors can prescribe treatments that may help one manage symptoms and feel better. Medicines can help one breathe easier. Most of the medications are inhaled so they go straight to the lungs. In time, a patient may need to use supplemental oxygen some or most of the time. People who have COPD are more likely to get lung infections, so patients will need to get a flu vaccine every year. The patient should also get a pneumococcal shot. It may not keep one from getting pneumonia, but if the patient does get pneumonia, the patient probably will not be as sick.

Medicines for COPD are used to: reduce shortness of breath, control coughing and wheezing, and prevent COPD flare-ups (i.e., exacerbations) or keep the flare-ups from being life-threatening. Most people with COPD find that medicines make it easier to breathe.

Some COPD medicines are used with devices called inhalers or nebulizers. Most doctors recommend using spacers with inhalers. It's important to learn how to use these devices correctly. Many people don't learn how to use these devices correctly, so they don't get the full benefit from the medicine.

Bronchodilators are used to open or relax the airways and help with shortness of breath. Short-acting bronchodilators ease the symptoms. They are considered a good first choice for treating stable COPD in a person whose symptoms come and go (intermittent symptoms). They include: anticholinergics (such as ipratropium), beta-2 agonists (such as albuterol and levalbuterol) and a combination of the two (such as a combination of albuterol and ipratropium). Long-acting bronchodilators help prevent breathing problems. They help people whose symptoms do not go away (persistent symptoms). They include: anticholinergics (such as tiotropium) and beta2-agonists (such as salmeterol, formoterol, and arformoterol).

Corticosteroids (such as prednisone) may be used in pill form to treat a COPD flare-up or in an inhaled form to prevent flare-ups. They are often used if you also have asthma. Other medicines include: Expectorants, such as guaifenesin (Mucinex), which may make it easier to cough up mucus. Doctors generally don't recommend using them. Methylxanthines, which generally are used for severe cases of COPD, may have serious side effects, so they are not usually recommended.

Lung surgery is rarely used to treat COPD. Surgery is never the first treatment choice and is only considered for people who have severe COPD that have not improved with other treatment. Surgery choices include lung volume reduction surgery which involves removal of part of one or both lungs, making room for the rest of the lung to work better. It is used only for severe emphysema; lung transplant: replaces a sick lung with a healthy lung from a person who has just died; and bullectomy which removes the part of the lung that has been damaged by the formation of large, air-filled sacs called bullae.

The embodiments of the present invention induce multiplication of pluripotent stem cells in situ, using the patient as their own sterile bioreactor to produce the desired quantities of stem cells without the potential for contamination and/or induction into other downstream cell types before their mobilization into the blood stream. The inventors have tested this concept in vivo in horses, showing an increase of 212% above normal and in vivo in humans, showing a steady increase in stem cell numbers based on the amount of subject composition ingested.

In one embodiment of the present invention, the composition is a blue-green algae known as Aphanizomenon flos-aquae (“AFA”) which is a freshwater species of cyanobacteria. AFA is marketed by Klamath Algae Products, Inc., dba E3Live located in Klamath Falls, Oreg. Those skilled in art will recognize that other plant-based cyanobacteria phytochemicals may be used as well. Cyanobacteria of any of a large group of prokaryotic, mostly photosynthetic organisms. Though classified as bacteria, they resemble the eukaryotic algae in many ways, including some physical characteristics and ecological niches. They contain certain pigments, which, with their chlorophyll, often give them a blue-green color, though many species are actually green, brown, yellow, black, or red. They are common in soil and in both salt and fresh water, and they can grow over a wide range of temperatures. Other compositions, including nutraceuticals or pharmaceuticals, such as Epogen, an injectable product to stimulate red blood cell production, Neupogen, an injectable product to stimulate white blood cell production, adaptogens (e.g., Protandim) may also provide an increase in pluripotent stem cell count. Thus, the use of AFA, or other compositions, including nutraceuticals or pharmaceuticals, allows for an ex vivo pluripotent stem cell population, the population having been generated in vivo in the mammal.

By establishing an ingestion protocol of AFA, the inventors have been able to increase the number of pluripotent stem cells (not to be confused with mesenchymal stem cells) in the subject's tissue and/or bloodstream. The pluripotent stem cells are a combination of epiblast-like stem cells (“ELSCs”), blastomere-like stem cells (“BLSCs”) and transitional cells.

Table 1 below details exemplary AFA oral ingestion protocols using 500 mg capsules of AFA for increasing the number of pluripotent stem cells in the subject's bloodstream.

TABLE 1

| Time Frame | Protocol |
|---|---|
| One Week | One capsule twice daily for two days; then |
| | Two capsules daily for two days; then |
| | Three capsules daily for two days; then |
| | Four capsules last day. |
| One Month | One capsule daily for one week; then |
| | Two capsules daily for one week; then |
| | Three capsules daily for one week, then |
| | Four capsules daily for one week. |
| Three Months | (a) One capsule daily for one month; then |
| | (b) Two capsules daily for one month; then |
| | (c) Three capsules daily for one month, then |
| | Four capsules morning before blood draw and repeat |
| | (a)-(c). |
| Seven Months | Follow one month protocol; then |
| (Includes 3 | (a) One capsule daily for one month; then |
| (regenerative | (b) Two capsules daily for one month; then |
| blood cell | (c) Three capsules daily for one month; then |
| (RBC) | Four capsules morning before blood draw and repeat |
| treatments)) | (a)-(c) for second and third RBC treatments. |
| Nine Months | (a) One capsule daily for one month; then |
| (Includes | (b) Two capsules daily for one month; then |
| 3 RBC | (c) Three capsules daily for one month; then |
| treatments) | Four capsules morning before blood draw and repeat |
| | (a)-(c) for second and third RBC treatments. |

Patients following an AFA ingestion protocol disclosed herein have shown large percentage increases in the number of pluriptent stem cells in vivo. In addition to the ingestion schedules detailed in Table 1, it is recommended that AFA be taken orally 90 or more minutes prior to a blood draw directed at harvesting as the pluripotent stem cell count peaks approximately 90 minutes after consumption.

The following paragraphs and flow chart 100 describe a procedure for harvesting pluripotent stem cells, re-constituting said pluripotent stem cells and infusing said pluripotent stem cells into a subject to treat various diseases. While the procedure is specific in some areas, it is understood that the procedure is exemplary in nature such that adjustments may be made within the spirit and scope of the embodiments of the present invention.

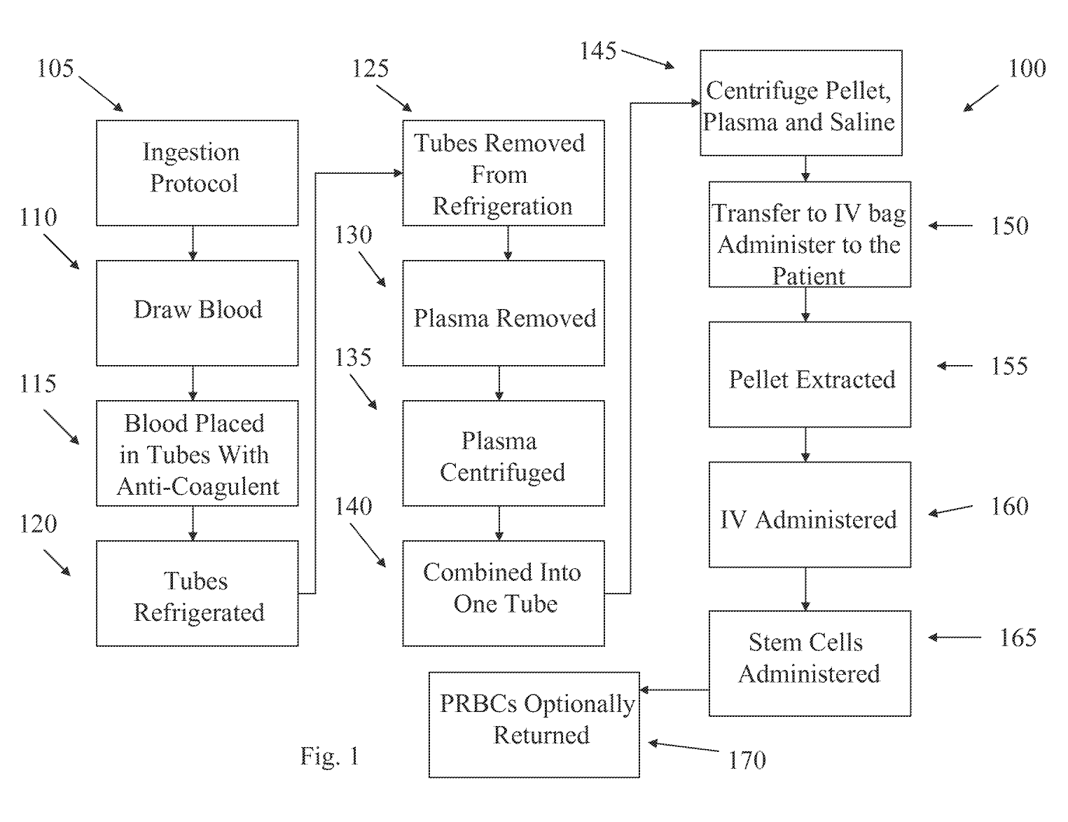

FIG. 1 shows a flow chart 100 of a procedure according to the embodiments of the present invention. Once the ingestion protocol or a portion thereof at 105 has lasted the desired time period, at 110, a venipuncture and blood draw are performed to collect 400 ml of blood from a peripheral vein using 4 ml and/or 10 ml Vacutainer® type tubes containing an anti-coagulant, such as ethylenediaminetetraacetic acid (EDTA), a 19-gauge butterfly needle and a luer adapter. Other anti-coagulants including citric acid and Heparin may also be used. At 115, after each tube is filled with blood it is shaken or inverted 4-5 times in order to mix it with the anti-coagulant and placed in a test tube tray or holder to maintain in an upright position.

At 120, the tray or holder with blood-filled tubes is then placed in a refrigerator at approximately 38 degrees Fahrenheit for 48 hours in order to allow a natural gravity separation to occur between the red blood cells and plasma. While 48 hours is a recommended time period, the tubes may remain longer in the refrigerated environment (e.g., 30 days) before pluripotent stem cells are harvested from the tubes.

At 125, the tubes are removed from the refrigerator and dried blood is cleaned from rubber tube stoppers using hydrogen peroxide and cotton. The stoppers are then cleaned using alcohol and cotton afterwhich the alcohol is allowed to dry. Prior to removing any plasma from the tubes, each stopper is punctured with a needle, such as an 18 gauge needle, to remove any vacuum remaining in the tube. In the alternative, a pipetter may be used and the stopper removed in order to remove plasma from the tubes. The latter should be conducted under sterile conditions performed under a flow hood and/or in a clean room with positive pressure and High-Efficiency Particulate Air (“HEPA”) filters. As much as possible, the user should also follow a clean or sterile technique using latex gloves, mask, goggles, gown, shoe coverings, etc., in order to avoid any contamination of the blood product(s).

At 130, plasma is removed from the upper half of the tubes using a syringe (e.g., 10 ml, 20 ml or 30 ml) and 18 gauge needle, 3 inches in length for an EDTA 10 ml tube and 2 inches for an EDTA 4 ml tube, to puncture the stopper. Plasma is removed from the tube via needle and syringe or via pipette and transferred into another container such as a 10 ml red top Vacutainer® tube without additive or 15 ml conical tube. This can be done in a few different ways as follows: (i) all of the plasma is removed and transferred to another tube for centrifuging; (ii) ⅓ of the upper plasma is removed and transferred to another tube for centrifuging; or (iii) ½ of the upper plasma is removed and transferred to another tube for centrifuging. Generally, a typical total yield of pluripotent stem cells from a 400 ml blood draw should be about 4-5 cc per tube or between 160 to 200 cc. Any remaining plasma is put into a 500 cc IV bag with 0.9% normal saline. For a 400 ml blood draw, approximately 200 cc may be withdrawn from the IV bag prior to adding any plasma.

At 135, all plasma in the tubes is centrifuged at about 5500 rpm for 5-15 minutes. The centrifuge may be at lesser or greater speeds (e.g., 4000 rpm) and the centrifuge time period (e.g., 20-60 minutes) may be more or less. This causes large pluripotent cells (a.k.a. ELSCs or epiblast-like stem cells), medium pluripotent cells (a.k.a. transitional cells) and small pluripotent cells (a.k.a. BLSCs or blastomere-like stem cells) to collect at the bottom of the tube and form a collection of cells or pellet. Any additional pluripotent cells, including ultra small cells requiring additional centrifuge time (e.g., 1 hour), that remain in the plasma are transferred into the IV bag. A small amount of plasma is left in each tube with the pellet. For example, a 15 ml tube will have approximately 13½ ml removed leaving 1½ ml in the tube. Each tube with a pellet and small amount of plasma is then either shaken against the operator's hand or placed on a shaker until the pellet has completely dissolved. At 140, all tubes with dissolved pellets are then transferred and combined into one tube. Additional 0.9% normal saline is then added to the one remaining tube with dissolved pellets filling the remainder of the tube. In the alternative, each tube can have 0.9% normal saline added to it individually as opposed to collectively combining them in one. At 145, the tube with pellet, plasma, and saline is then centrifuged for 5-15 minutes to wash the pluripotent stem cells and free them of any immunoglobulins.

At 150, after centrifuging, the remaining plasma and 0.9% normal saline solution is then transferred into the IV bag and administered to the patient. It is best for maximum cell count (e.g., 1-5 billion total cells) for the plasma and pellet to be returned to the patient/subject the same day on which the separation occurs.

At 155, the remaining pellet is extracted via small syringe (e.g., 3 cc or 5 cc) with a 2 or 3 inch 18 gauge needle or via pipette. Any remaining pellet and/or packed red blood cells (“PRBC”) not extracted may optionally be reconstituted with small amount of 0.9% normal saline and placed into the IV bag. At 160, the mixture of pluripotent stem cells and 0.9% normal saline IV bag is administered to patient via intravenous drip infusion at a drip rate of anywhere from 60 drops per minute or less to wide open according to patient tolerance until entire contents of IV bag have been infused.

At 165, the pellet may then be used in any of the following ways: (a) Nebulization; (b) Intravenous bolus; (c) Intranasal inhalation; (d) Intra-spinal injection; (e) Intra-articular injection; (f) Topical cream; and/or (g) Eye drops. Each infusion technique is described in detail below.

Nebulization involves generally: (a) dissolving pellet in about 3 ml 0.9% normal saline; (b) adding mixture to nebulizer; and (c) nebulizing. More specifically, nebulizing involves: (a) centrifuging at setting about 5,500 times gravity to spin the tube for 5-15 minutes; (b) pouring off plasma (including immunoglobulins); (c) adding about 10 ml 0.9% normal saline to the remaining solid or dry pluripotent stem cells; (d) shaking to wash pluripotent stem cells thoroughly; (e) centrifuging for about 5-15 minutes at no more than about 5,500 times gravity; (f) pouring off liquid; (g) adding an adequate amount (e.g., 3-5 ml) 0.9% normal saline to the remaining solid or dry pluripotent stem cells; (h) shaking to reconstitute pluripotent stem cells thoroughly; (i) adding mixture to nebulizer; and (j) nebulizing.

Intravenous bolus involves: (a) dissolving pellet in small amount 0.9% normal saline and injecting via slow intravenous push; and (b) following with IV bag. More specifically, (a) adding plasma from sterile tube to 500 cc 0.9% normal saline; and (b) running intravenous infusion at approximately 120 drops per minute.

Intra-nasal inhalation involves: (a) dropping pellet into the nasal cavity of patient in Trendelenburg position (e.g., supine position with head lower than feet); and (b) keeping the patient in this position for 5-10 minutes. This procedure may be same as that described relative to nebulization, except that instead of nebulization the resulting solution is dripped into the nasal cavity with patient in a Trendelenburg position for 5-10 minutes. It is anticipated that intra-nasal inhalation may also be appropriate for children, such as those with Autism, because of the simplicity of the approach.

Intrathecal injection involves: (a) extracting spinal fluid from the lumbar cistern with a lumbar puncture needle (e.g., 23 gauge, 3½ inches); and (b) replacing equal amount of fluid withdrawn with pellet dissolved in 0.9% normal saline. In another embodiment, (a) extracting spinal fluid from the lumbar cistern with a lumbar puncture needle (e.g., 23 gauge, 3½ inches), (b) mixing the spinal fluid with the pluripotent stem cells, instead of 0.9% saline, and reintroducing the same amount of spinal fluid, but now with mixed cells, back into the spinal canal.

Intra-articular/Intra-muscular injection involves: (a) dissolving pellet in small amount of plasma (previously set aside and withheld from IV bag); (b) mixing with an equal amount of anesthetic (e.g., Marcaine 0.5%, Procaine 1%, Lidocaine 1%, etc.); and (c) injecting into joint and/or into area surrounding where soft tissue structures are located and/or attached (e.g., tendons, ligaments, cartilage, etc.).

Topical cream involves: (a) putting dissolved pellet solution into topical cream (e.g., lipophilic base); and (b) applying cream locally to area of interest (e.g., eczema, injury, burn, etc.).

Eye drops involves: (a) dissolving pellet in 0.9% normal saline; (b) adding small amount dimethyl sulfoxide (DMSO) (e.g., 0.1 to 0.2 cc); and (c) dropping at intervals into the affected eye(s).

Stereotactic procedures may also be used to infuse the pluripotent stem cells into the patient/subject.

After the IV and pellet administration have been accomplished, at 170, the packed red blood cells (“PRBC”) remaining in the EDTA tubes may be either discarded or optionally returned to patient as follows: (a) putting PRBC into an IV bag with 0.9% normal saline (e.g., 500 cc bag from which 200 cc were removed); and (b) optionally adding Heparin (e.g., 1000 IU); and/or optionally adding H₂O₂ 0.0375% (e.g., 2.5 to 3.0 cc); and/or passing IV bag through ultraviolet light for irradiation of PRBC. In this manner, everything removed from the patient during the blood draw may be placed back into the patient.

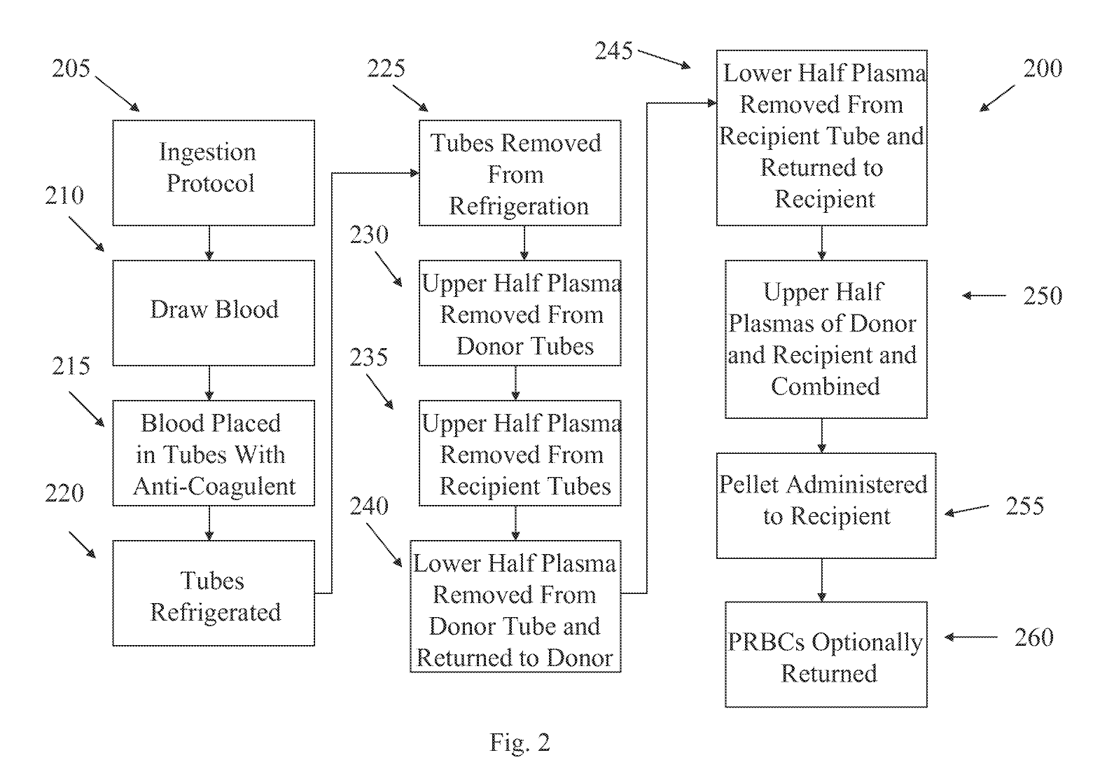

For allogenic use, the pluripotent stem cells may be extracted from blood of one person (“donor”) and administered for another person (“recipient”) so long as they both are the same gender and same blood type. For example, if recipient has a suspected or known DNA or inherited defect for which recipient's own pluripotent stem cells may be inadequate to repair. FIG. 2 shows a flow chart 200 describing a procedure for harvesting pluripotent stem cells, re-constituting said pluripotent stem cells and infusing said pluripotent stem cells into a recipient to treat various diseases. Steps 205-225 correspond to steps 105-125 of flow chart 100. At 230, upper half of plasma is removed from donor tubes as described in step 130 of flow chart 100 (see, paragraph [0043]). At 235, upper half of plasma is removed from recipient tubes as described in step 130 of flow chart 100. At 240, lower half of plasma is removed from donor tubes as described in step 130 of flow chart 100 and returned to donor as described in step 150 of flow chart 100 (see, paragraph [0045]). At 245, lower half of plasma is removed from recipient tubes as described in step 130 of flow chart 100 and returned to recipient as described in step 150 of flow chart 100. At 250, upper half of plasma from donor and recipient tubes in steps 230 and 235 are combined and processed as described in steps 135-160 for recipient use (see, paragraphs [0044]-[0046]). At 255, the pellet obtained in step 250 may be used for recipient in any of the following ways: (a) Nebulization; (b) Intravenous bolus; (c) Intranasal inhalation; (d) Intra-spinal injection; (e) Intra-articular injection; (f) Topical cream; and/or (f) Eye drops. Each infusion technique is described in detail below. At 260, remaining PRBCs may optionally be returned to respective donor or recipient as described relative to step 165 of flow chart 100 (see, paragraph [0055]).

Side effects normally associated with using stem cells from a donor with a different recipient are minimized by: (i) using patients with the same blood type (with blood transfusions, it is possible that those with blood type O and Rh negative may be a universal donor for pluripotent stem cells as well); (ii) using patients with same gender; (iii) using upper half of plasma from donor patient to obtain the small and medium or transitional pluripotent stem cells and then combining with the upper half of the recipient patient's plasma; (iv) generating a pellet from the combination of upper half of serum from both patients with the remaining plasma used in combination with 0.9% normal saline for treatment of the recipient patient via intravenous infusion per protocol. The pellet can be used per protocol for treatment of the recipient patient's respective condition(s) in any of the aforementioned methods (e.g., intra-nasal, intra-articular, intrathecal, intravenous, etc.). The lower half of the plasma from the recipient patient is used for treatment of the same or recipient patient via intravenous infusion and the lower half of the plasma from the donor patient is used for treatment of the same or donor patient primarily via intravenous infusion, but may be used to generate a pellet as well with remaining plasma used in combination with 0.9% normal saline for treatment of the recipient patient via intravenous infusion per protocol. If necessary (e.g., patient has anemia, iron deficiency, weakness, etc.), the autologous regenerated blood cells may be returned to the same patient as well.

Table 2 below lists exemplary diseases and infusion method used to treat the same.

TABLE 2

| Infusion Protocol | Disease |
|---|---|
| Nebulization | COPD, emphysema, pulmonary fibrosis, asthma |
| Intravenous | Systemic Conditions (e.g., chronic fatigue |
| | syndrome, fibromyalgia) |
| | Organ Specific Diseases (e.g., diabetes, |
| | congestive heart failure, cardiomyopathy, kidney |
| | diseases, liver diseases) |
| | Autoimmune Diseases (e.g., arthritis, lupus, MS, |
| | Hashimoto's thyroiditis) |
| Intranasal Inhalation | Neurological (Brain) Disorders (e.g., |
| | Parkinson's, Alzheimer's, ALS, MS, autism) |
| Intra-Spinal Injection | Neurological (Spine) Disorders (e.g., MS, spinal |
| | cord injuries) |
| Intra-Articular | Joint Disorders (e.g., joint injuries, |
| Injection | chondromalacia, arthritis) |
| Topical Cream | Skin Disorders (e.g., eczema, burns, wounds) |
| Eye Drops | Eye Disorders (e.g., macular degeneration) |

In another embodiment, said pluripotent cells are processed into freeze-dried pluripotent cells (“FDPCs”). In such an embodiment, said FDPCs are rehydrated, cultivated and differentiated into at least two separate pluripotent cell sizes in vitro, such as epiblast-like stem cells (“ELSCs”) and blastomere-like stem cells (“BLSCs”). The ELSCs and BLSCs or said separate pluripotent cells sizes may be freeze-dried and processed into dessicated pluripotent cells (“DPCs). Reconstituting is accomplished with an appropriate amount of normal saline 0.9% solution and reintroduced to an autologous body via any appropriate means such as intravenous infusion, nebulization, intrathecal injection, intramuscular injection, intra-articular injection or intra-nasal inhalation. Said pluripotent stem cells are reconstituted with an appropriate amount of the saline solution and introduced to an allogenic body of the same sex or said pluripotent cells are reconstituted with an appropriate amount of the saline solution and mixed with autologous stem cells before being introduced to an allogenic body of the same sex.

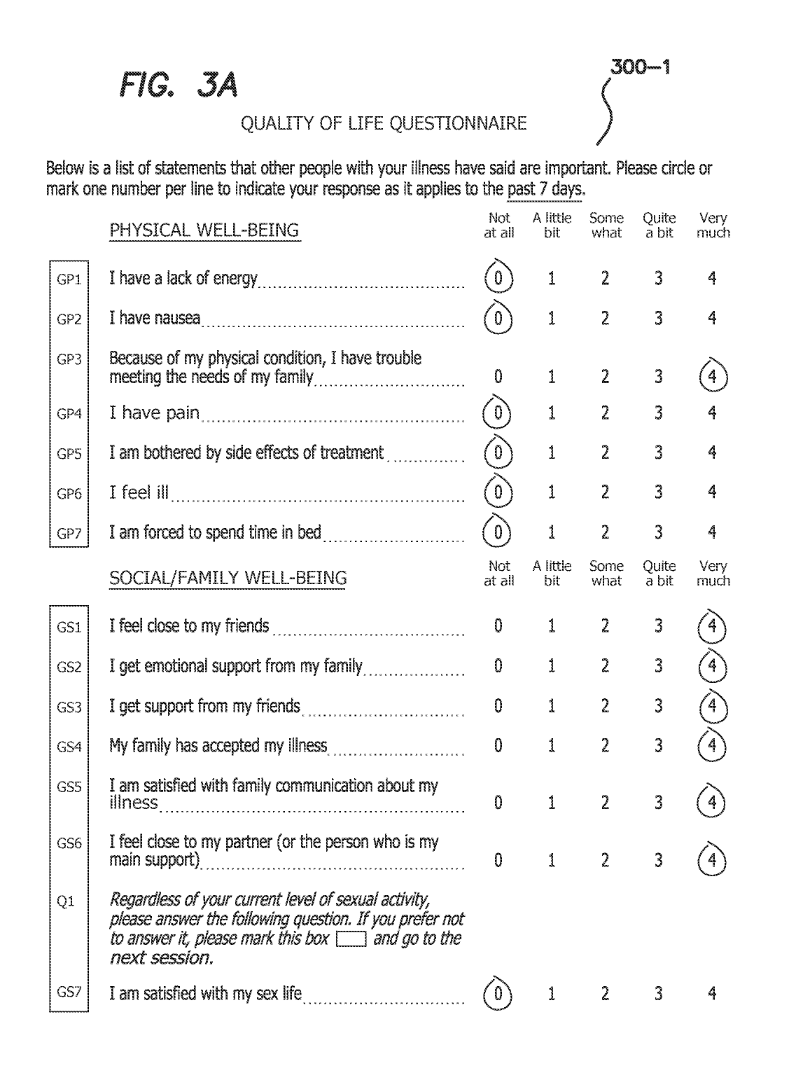

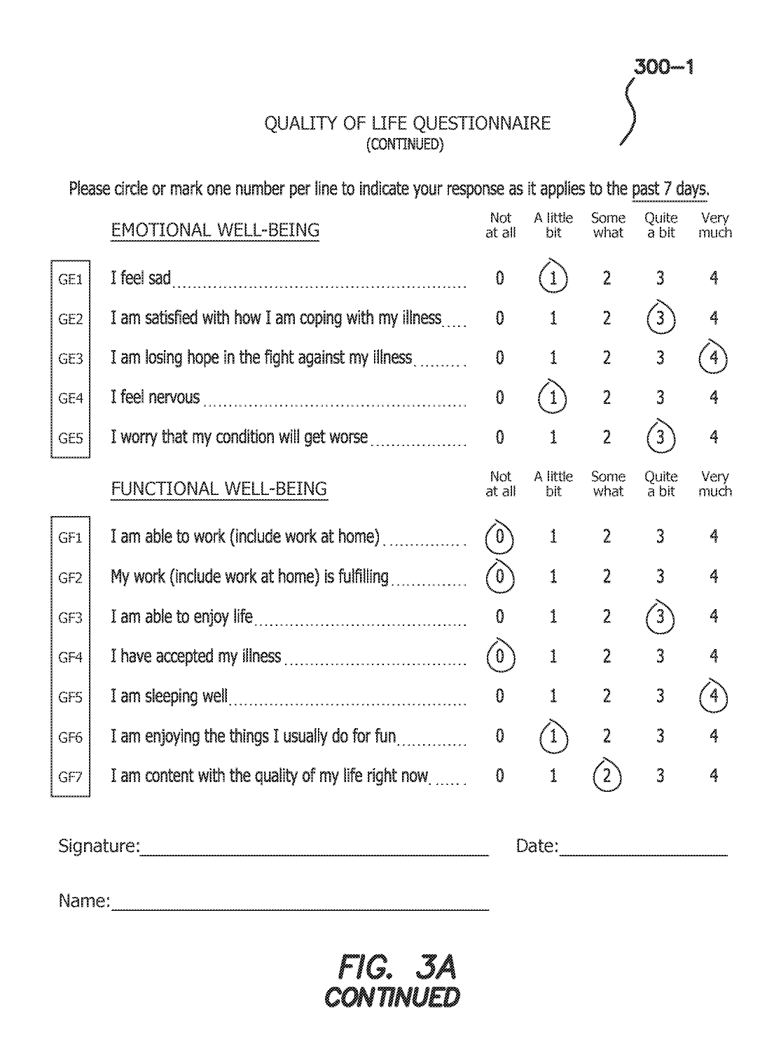

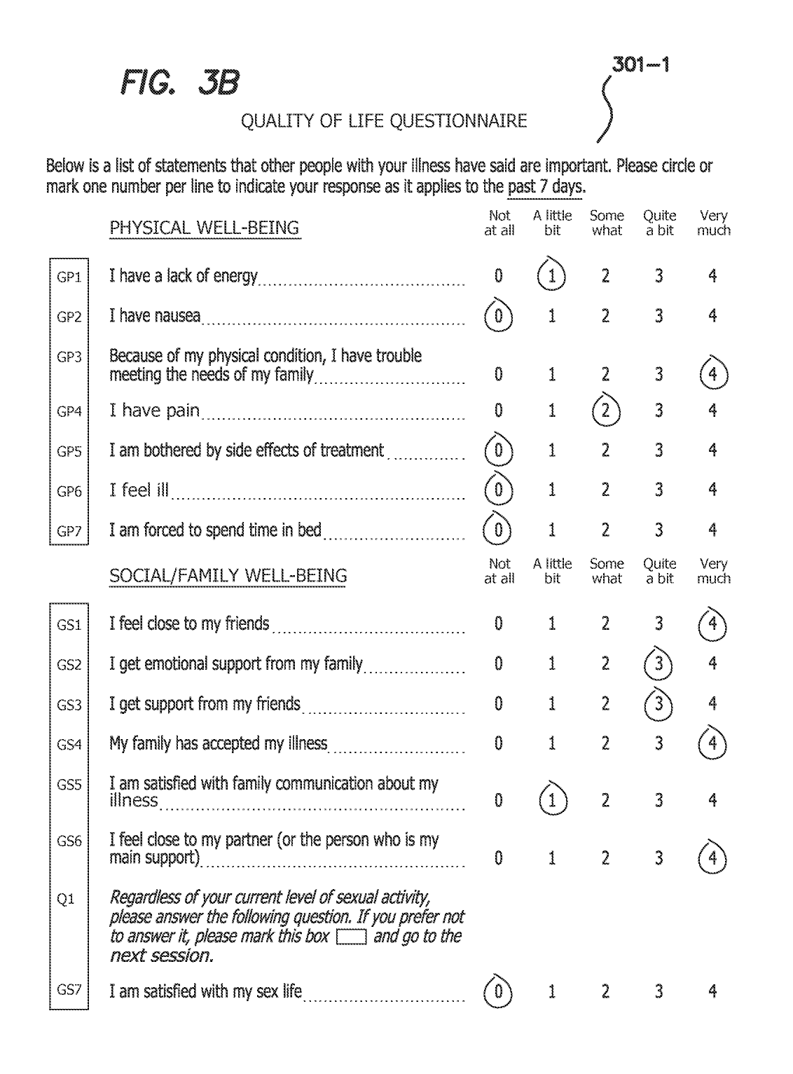

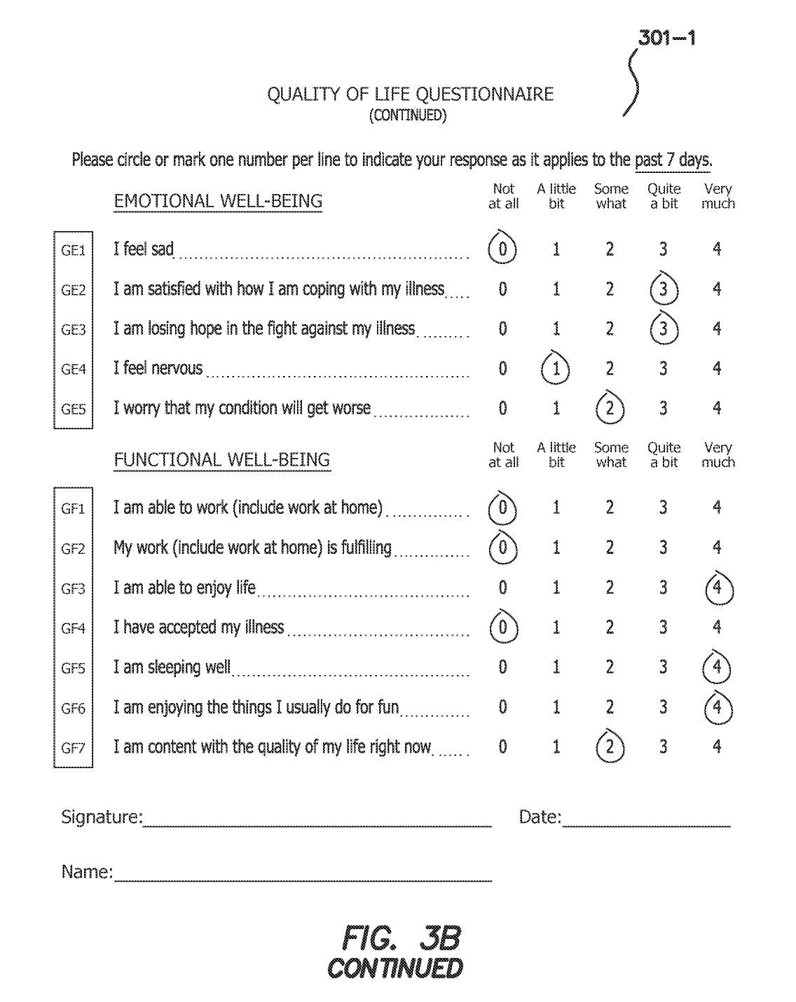

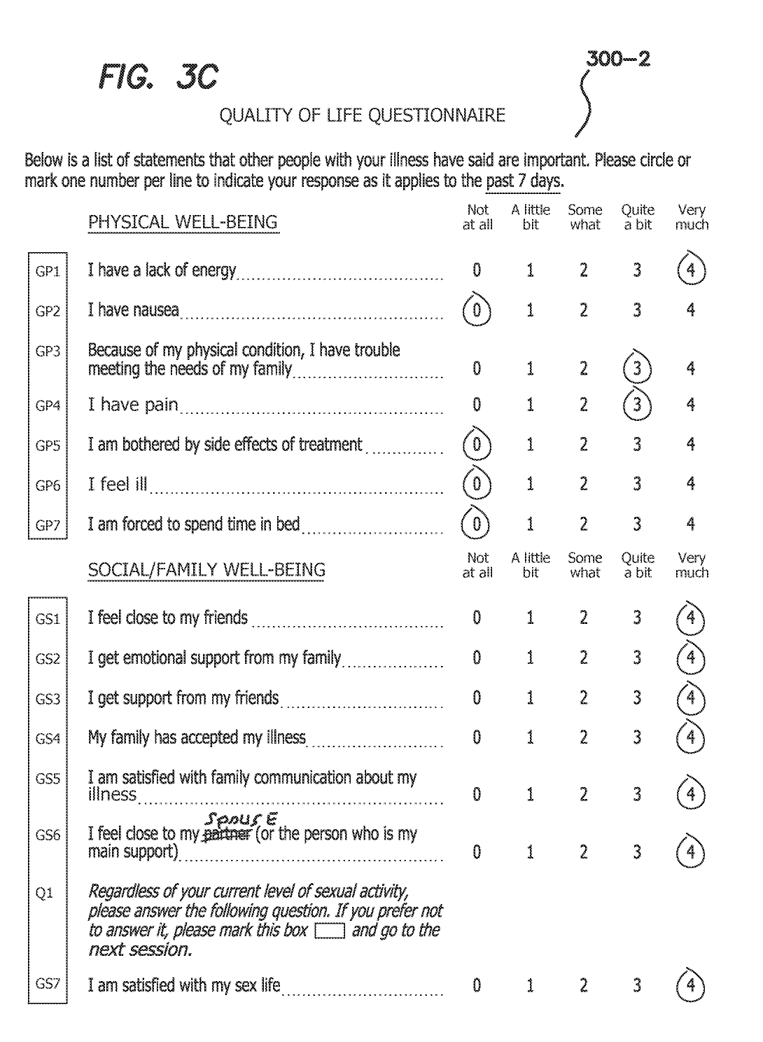

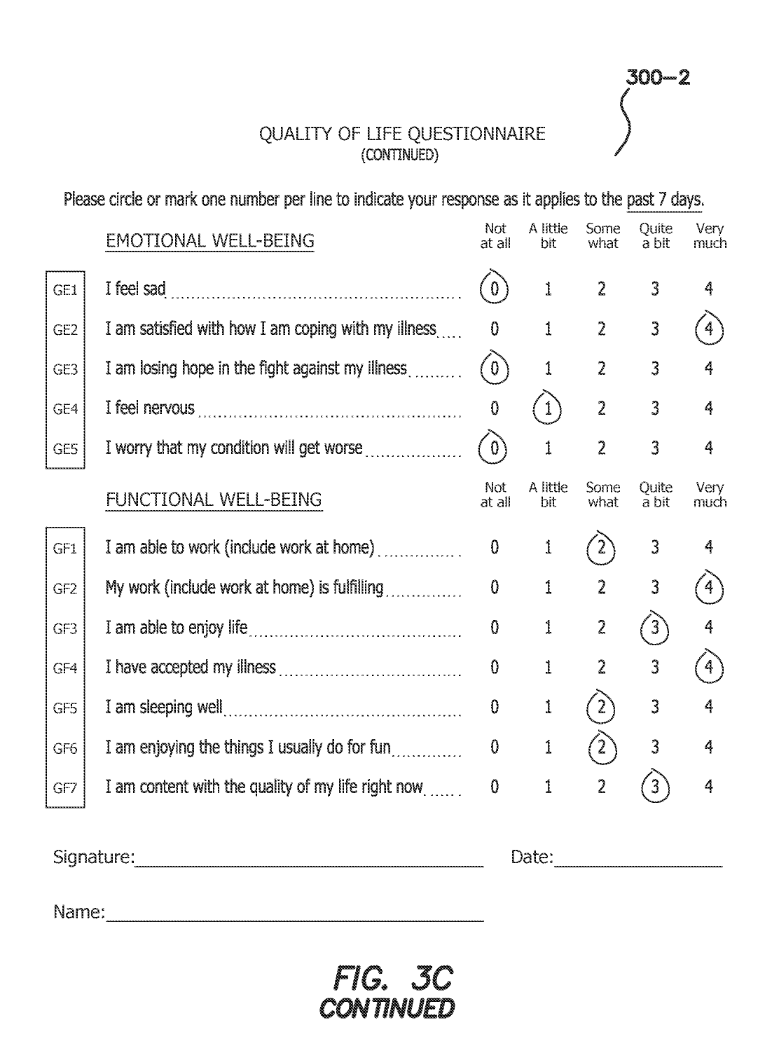

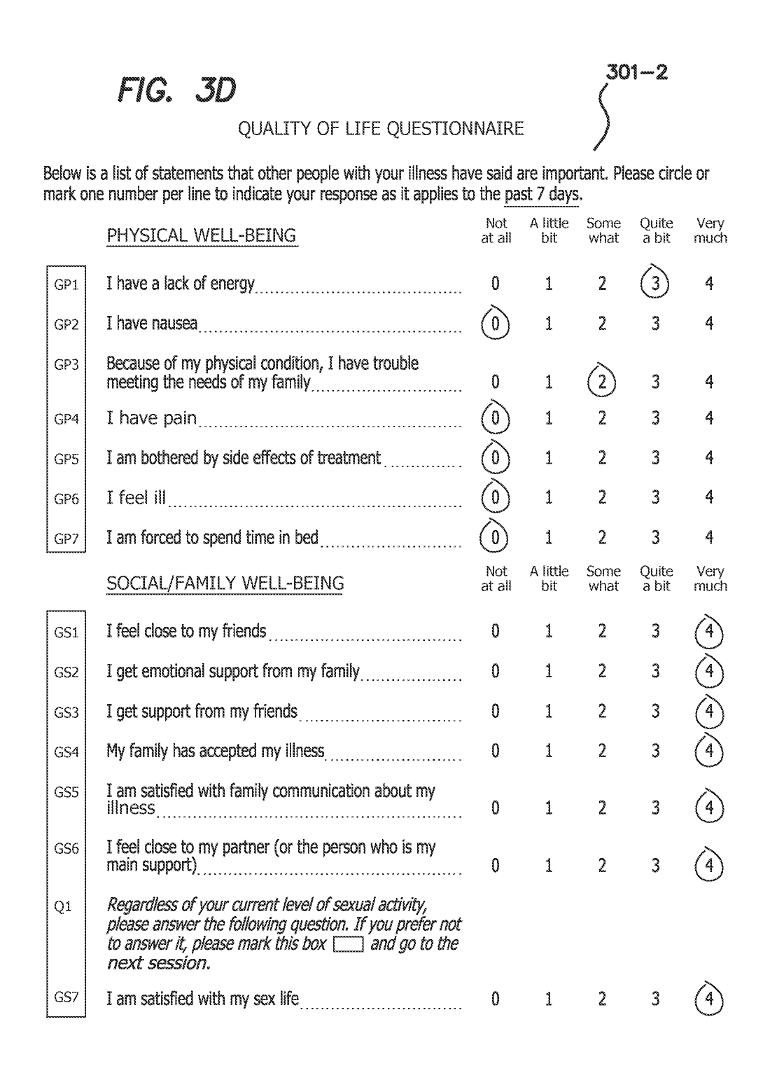

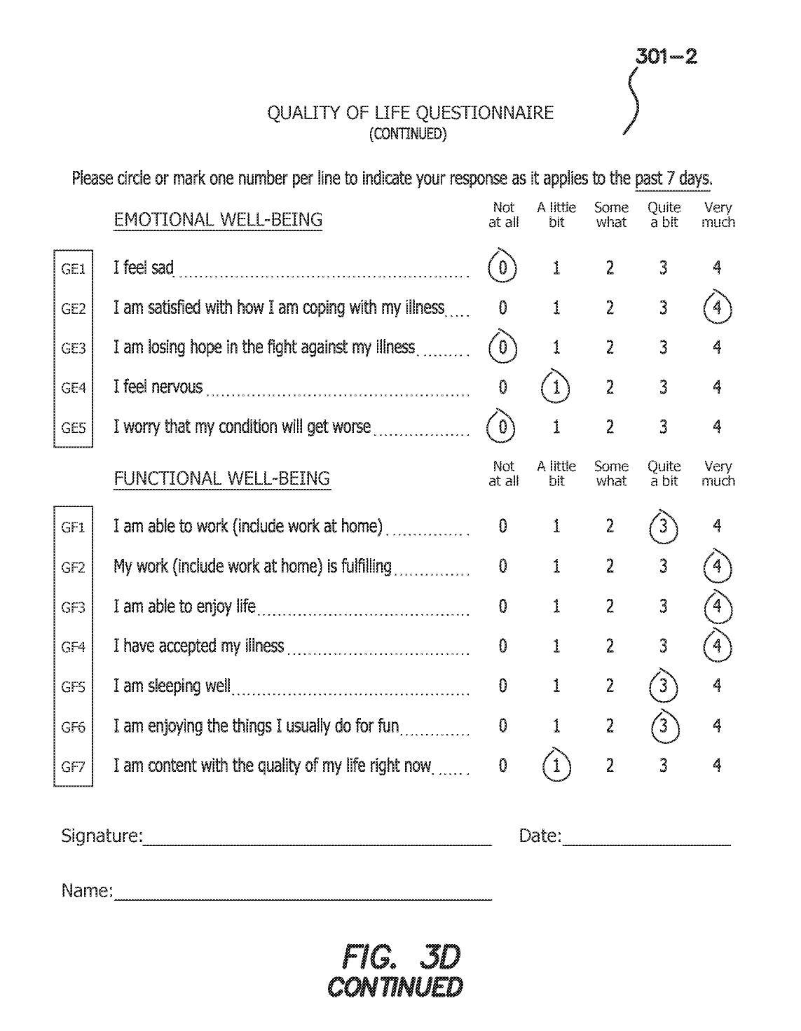

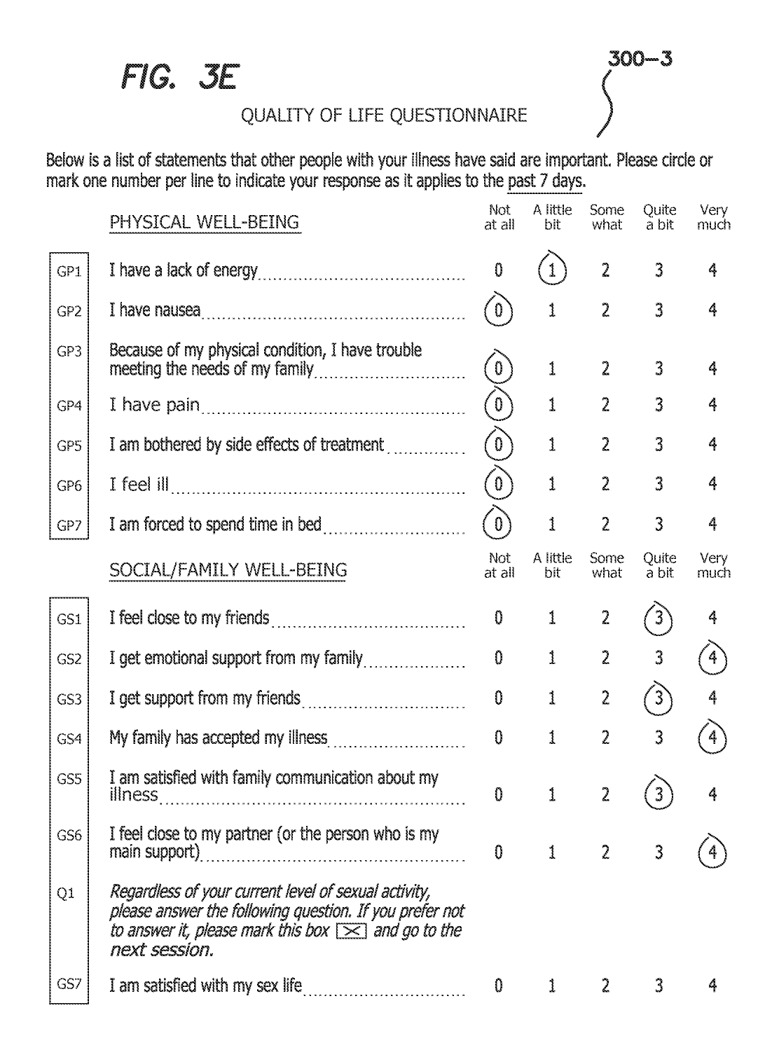

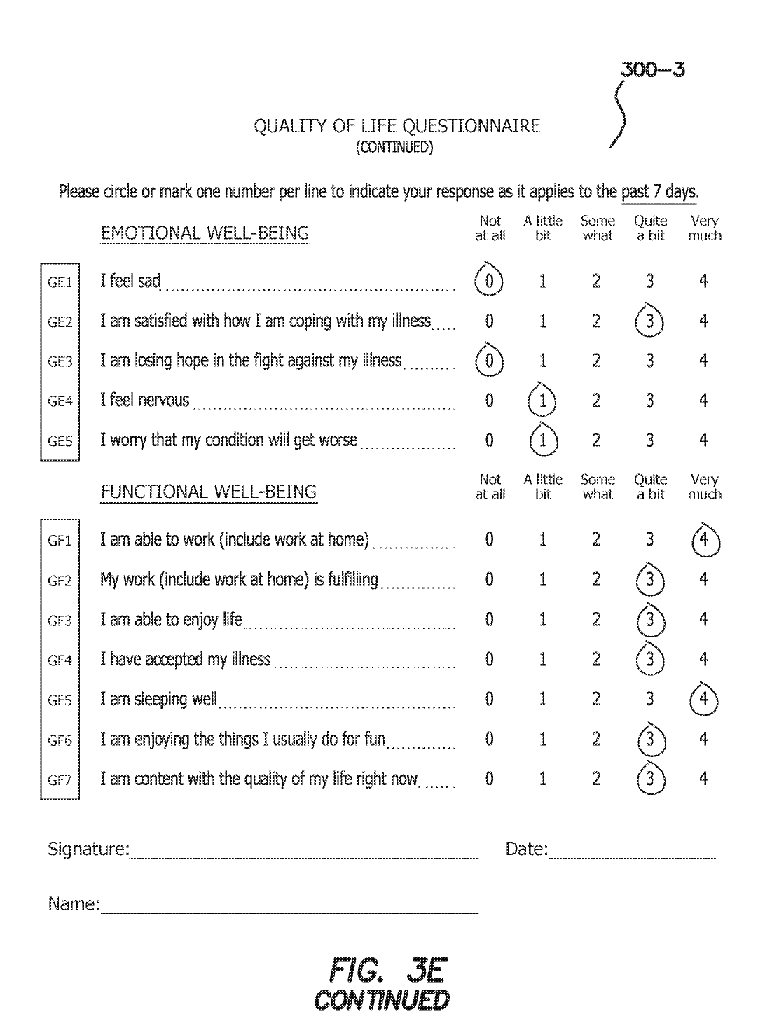

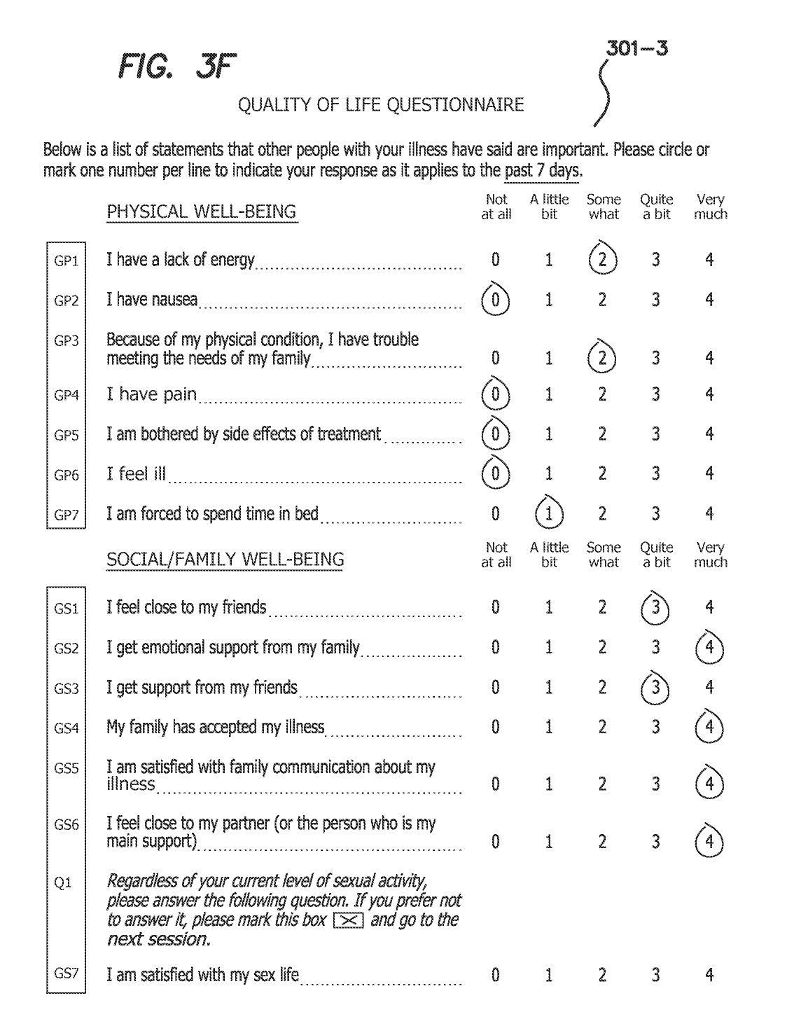

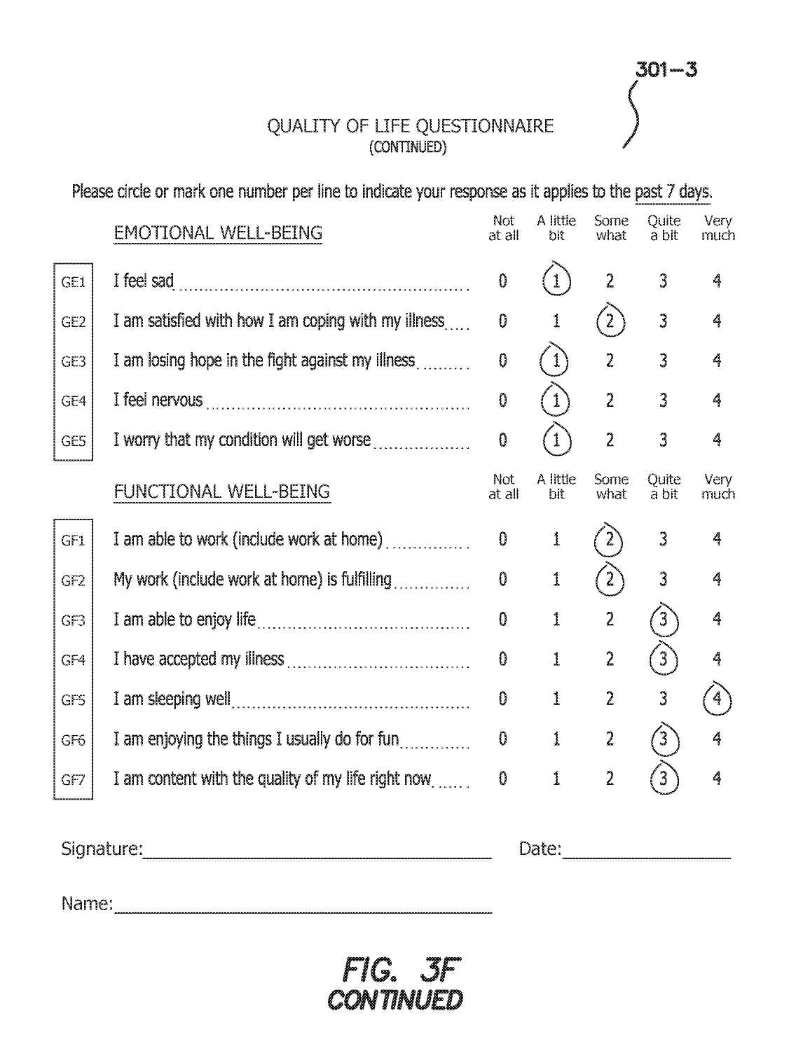

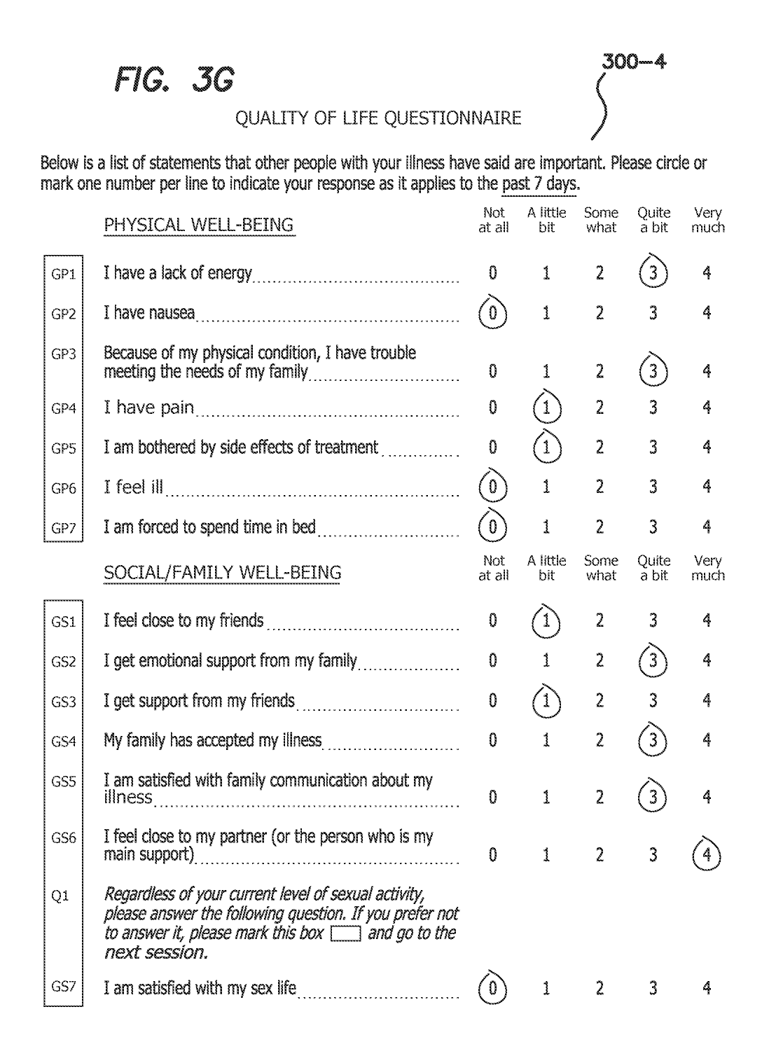

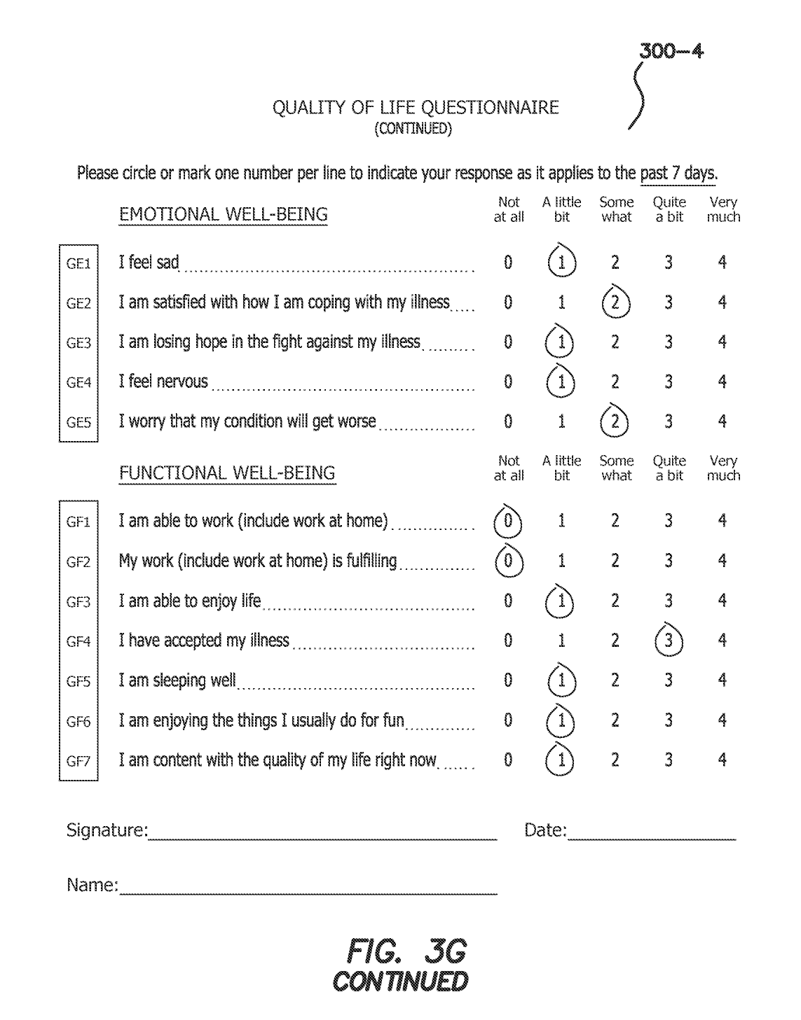

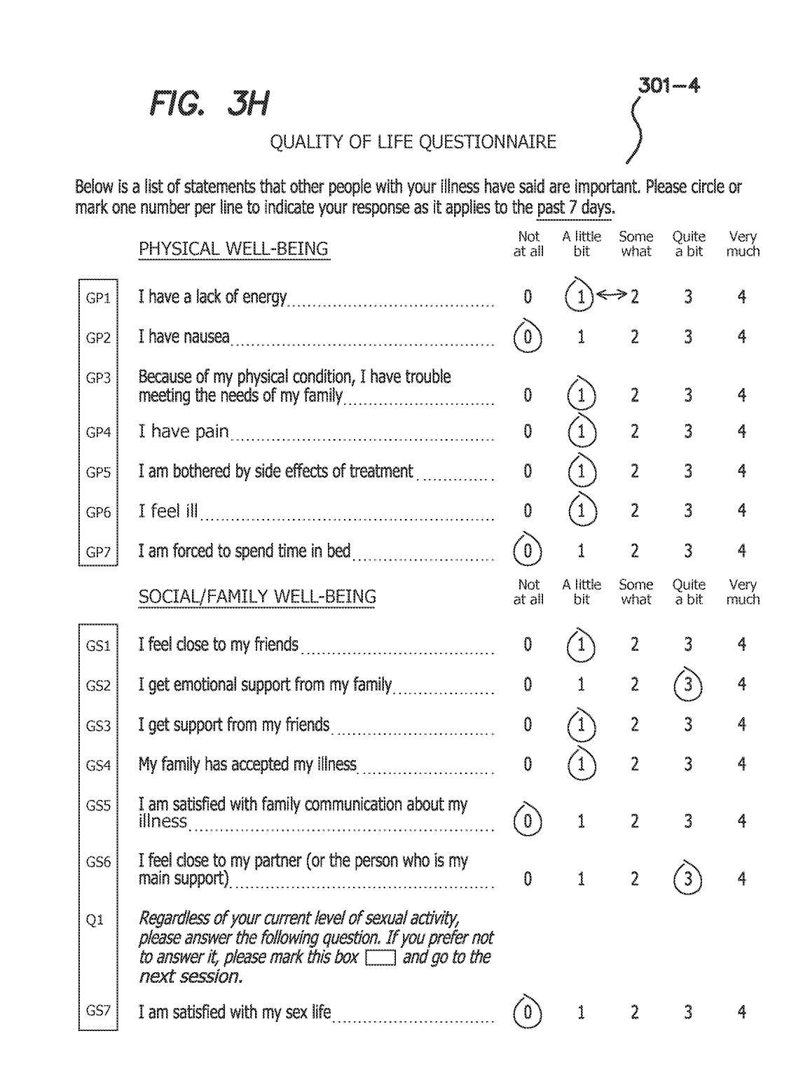

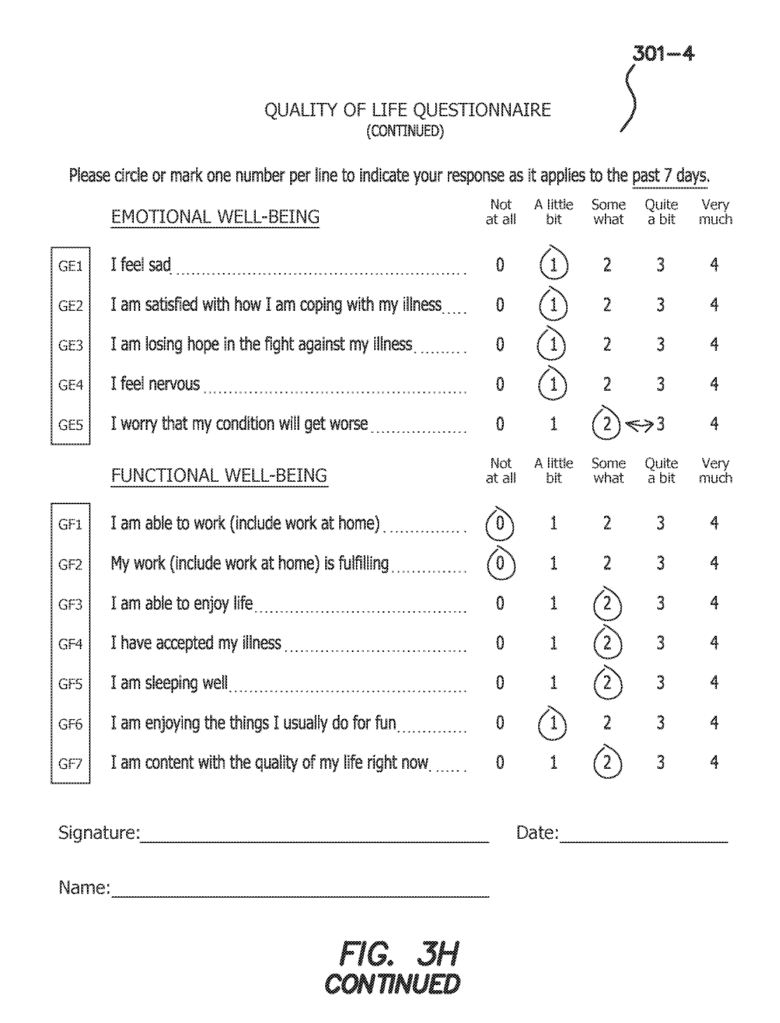

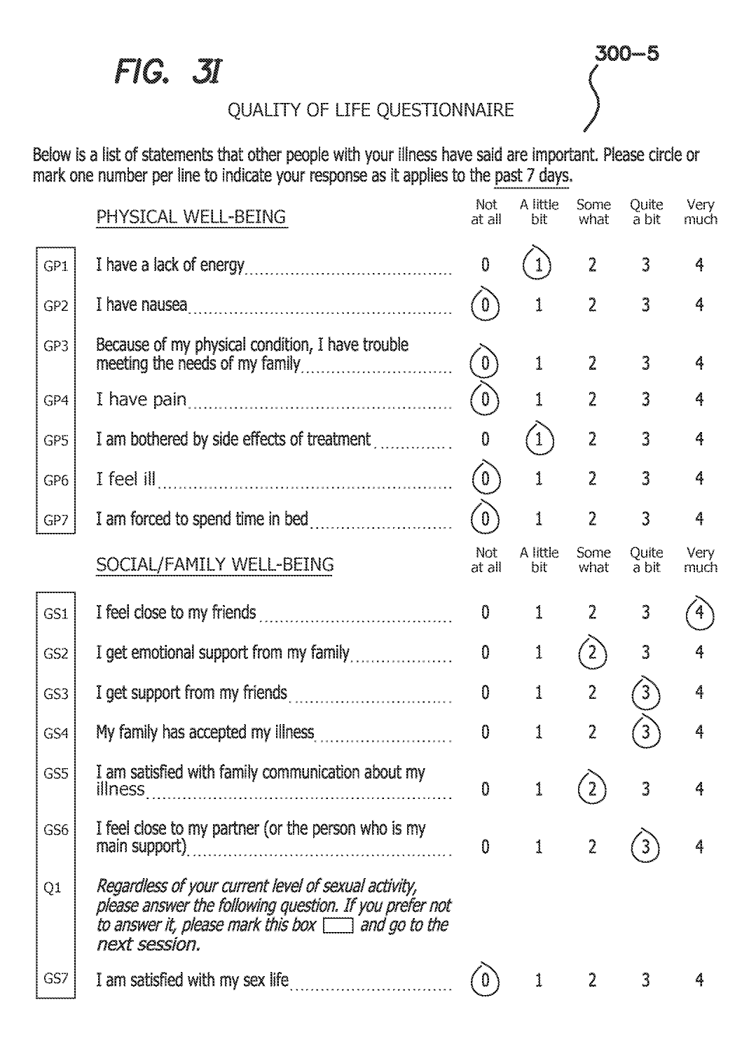

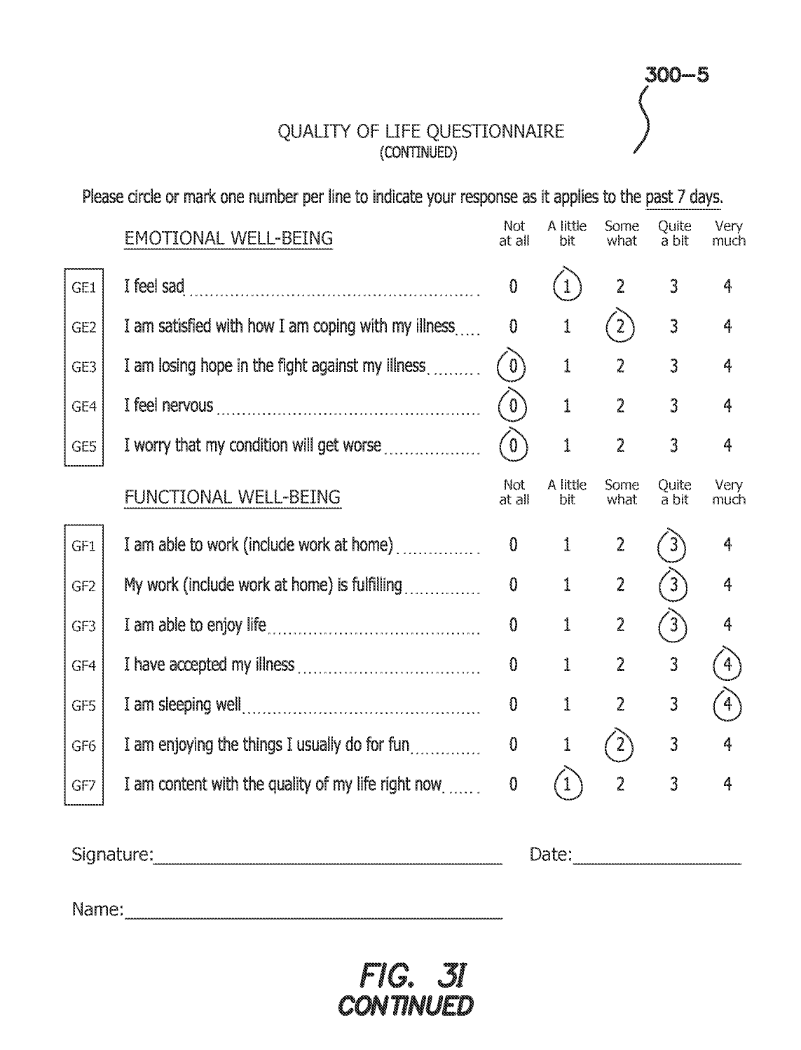

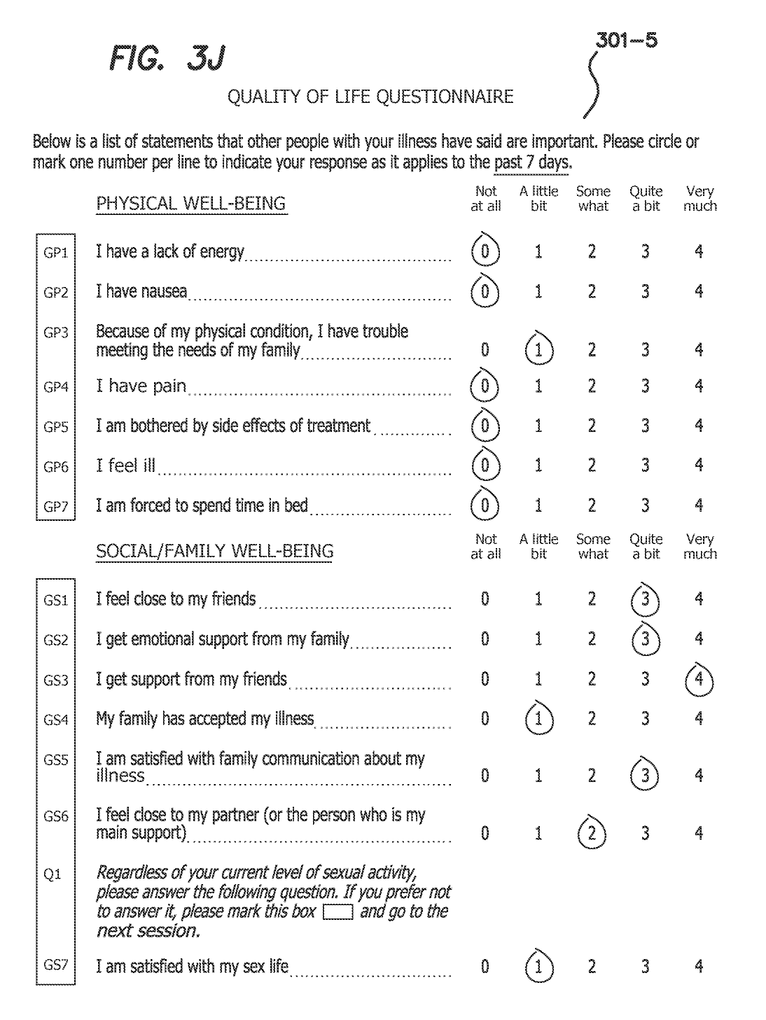

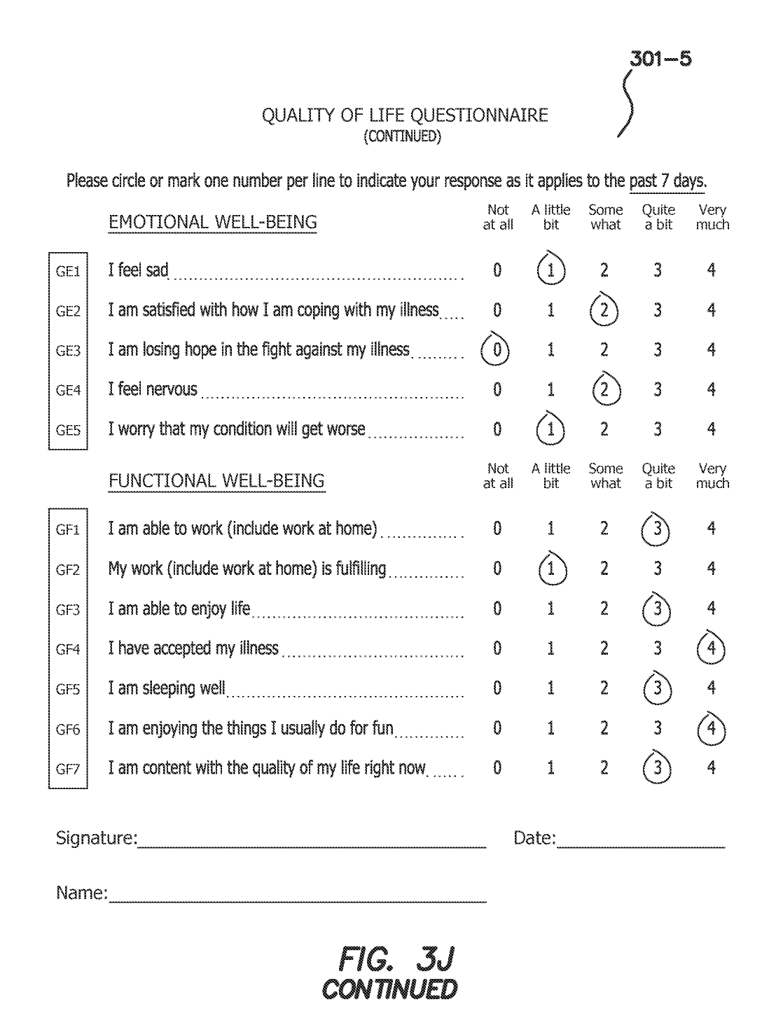

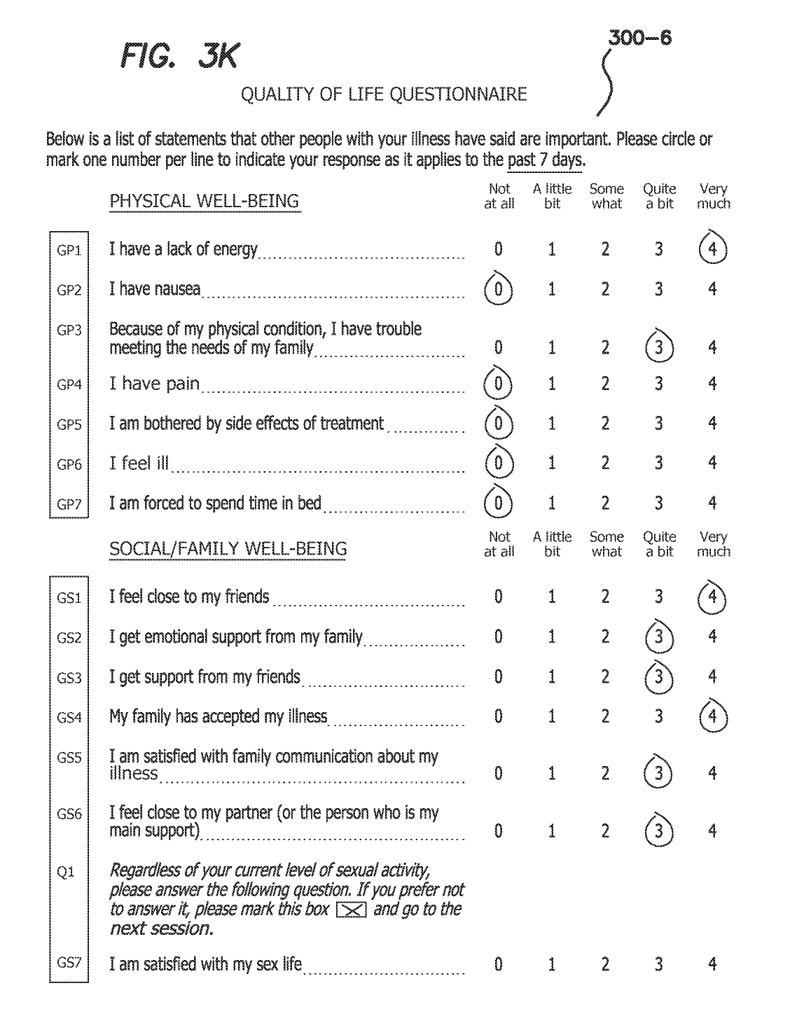

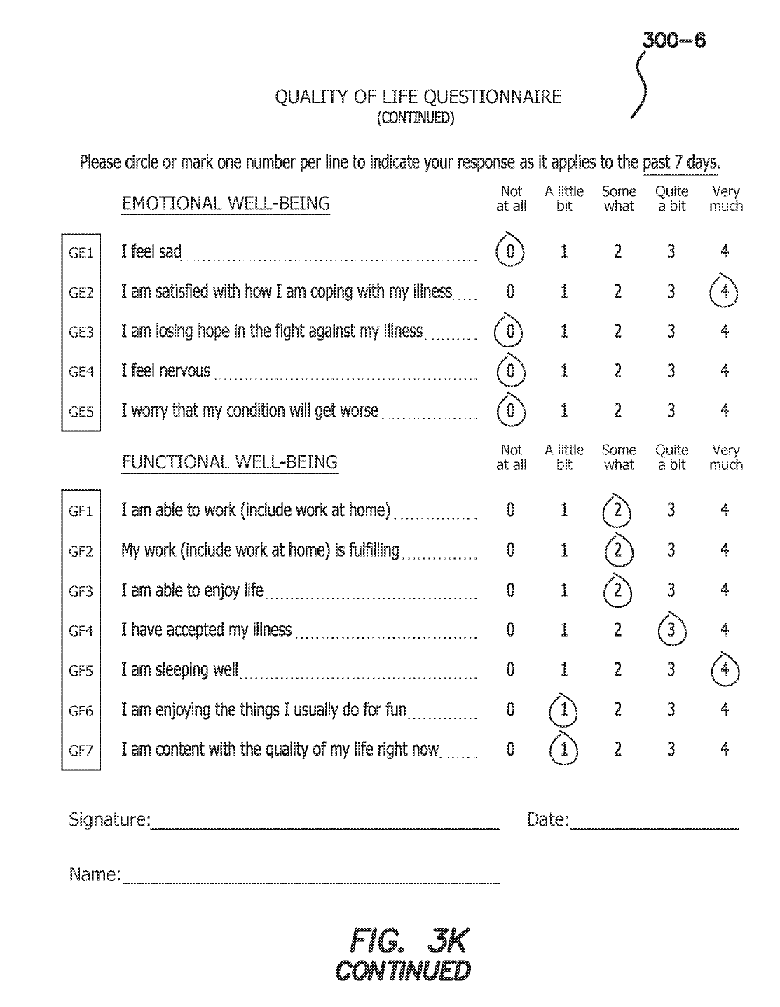

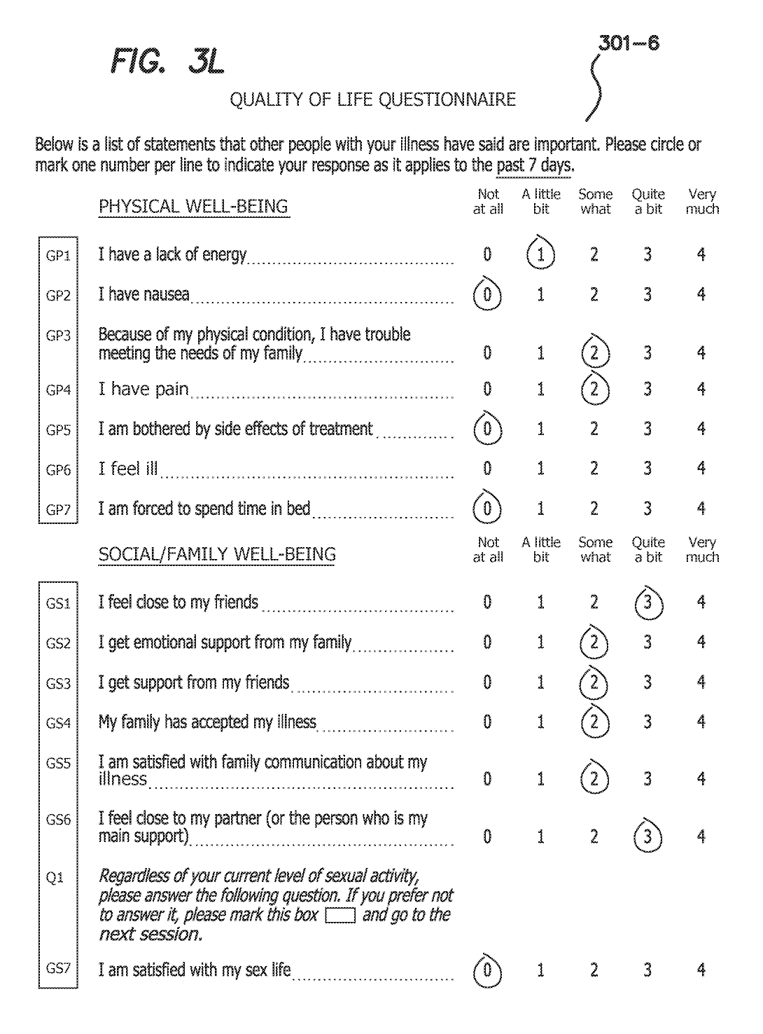

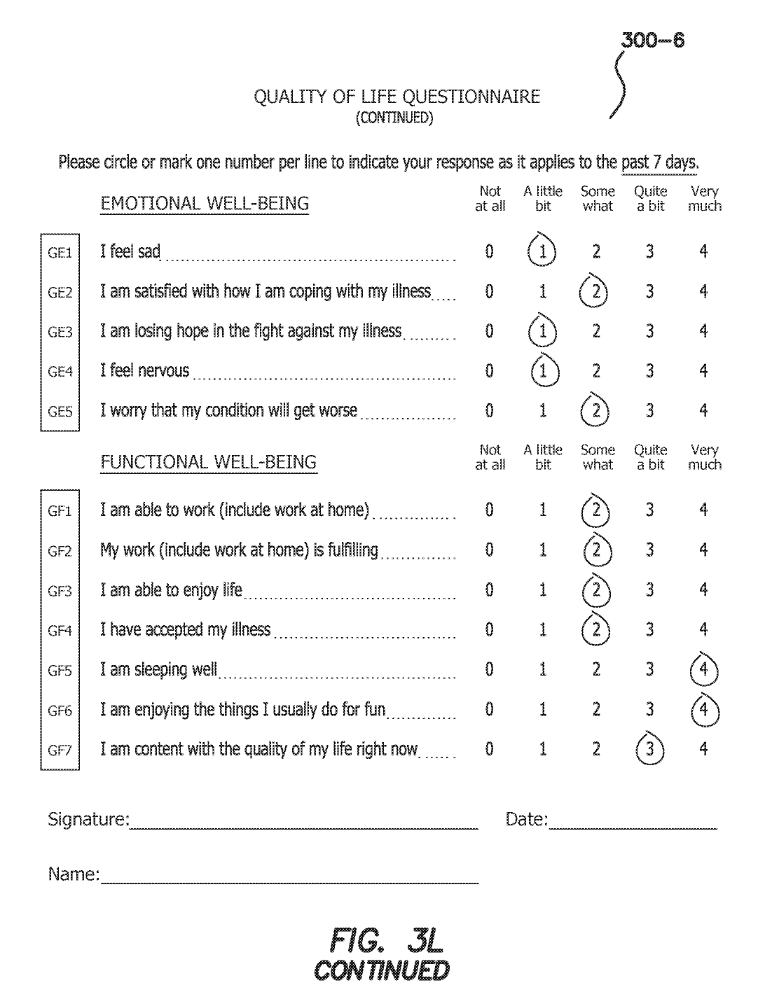

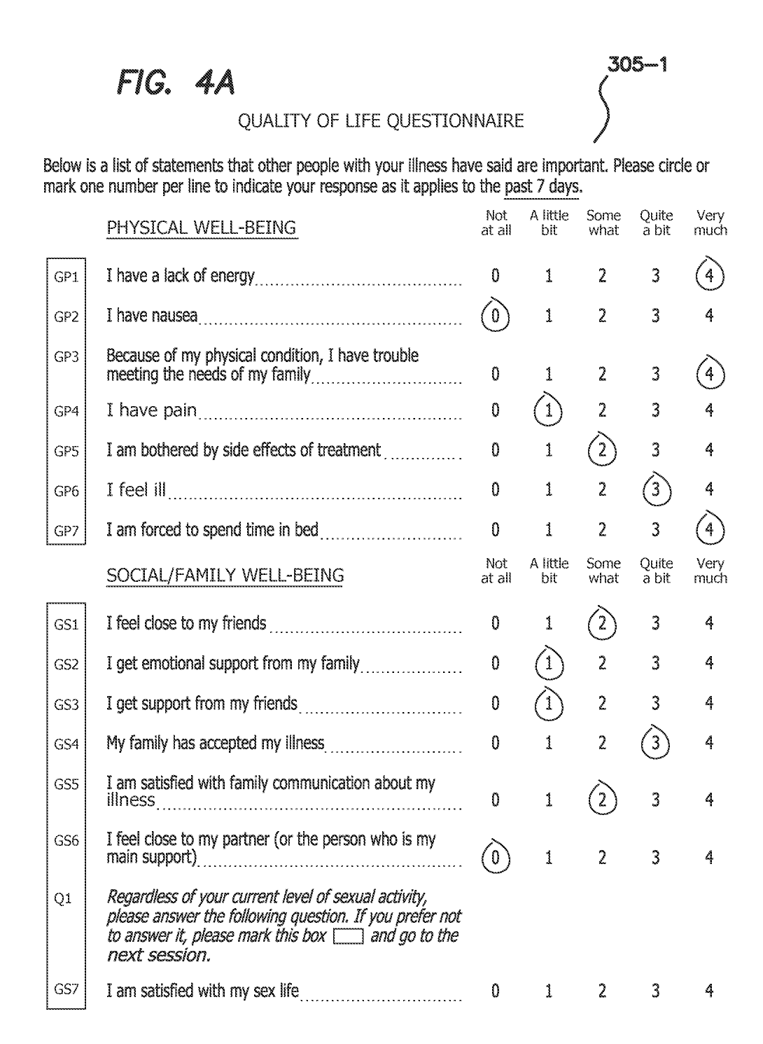

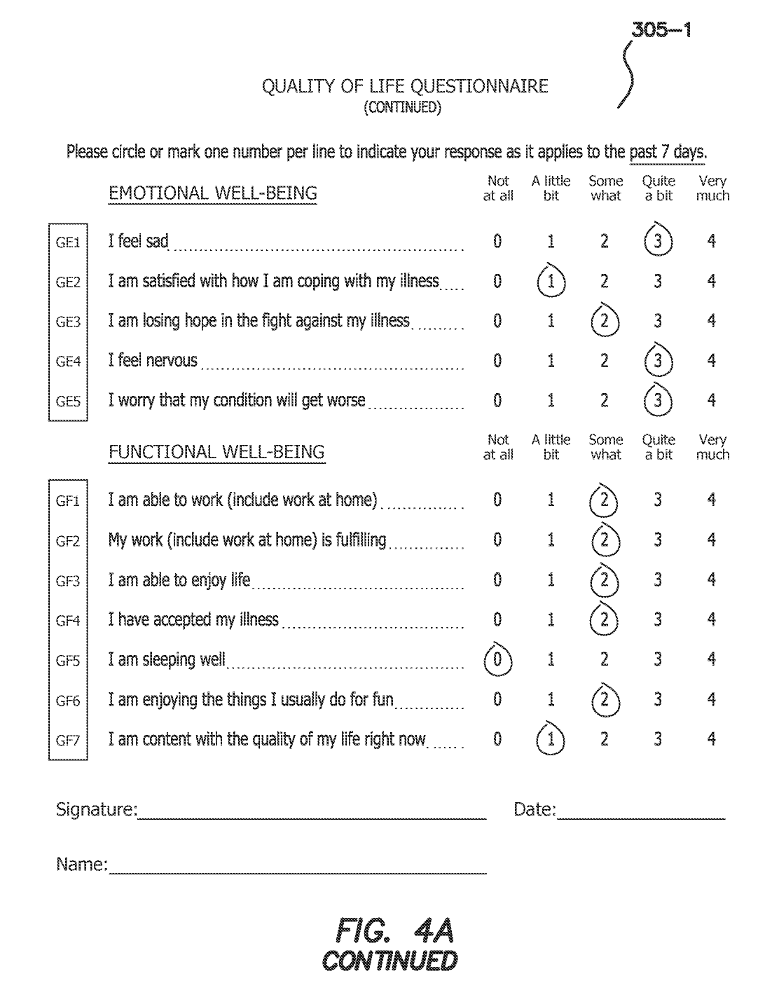

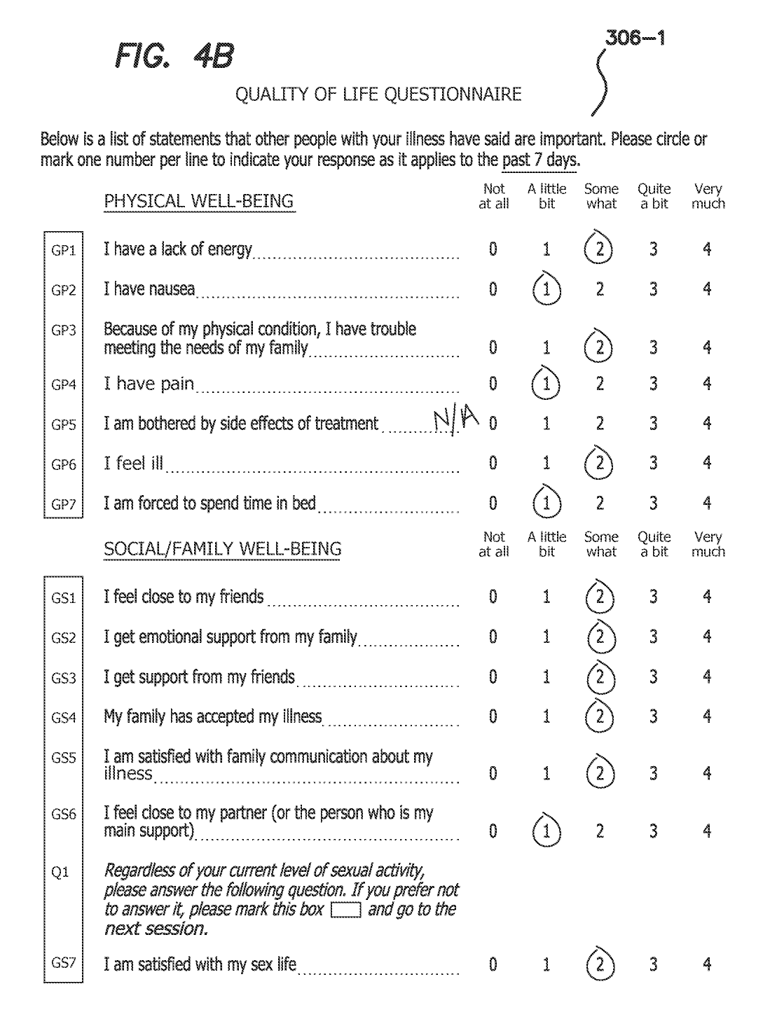

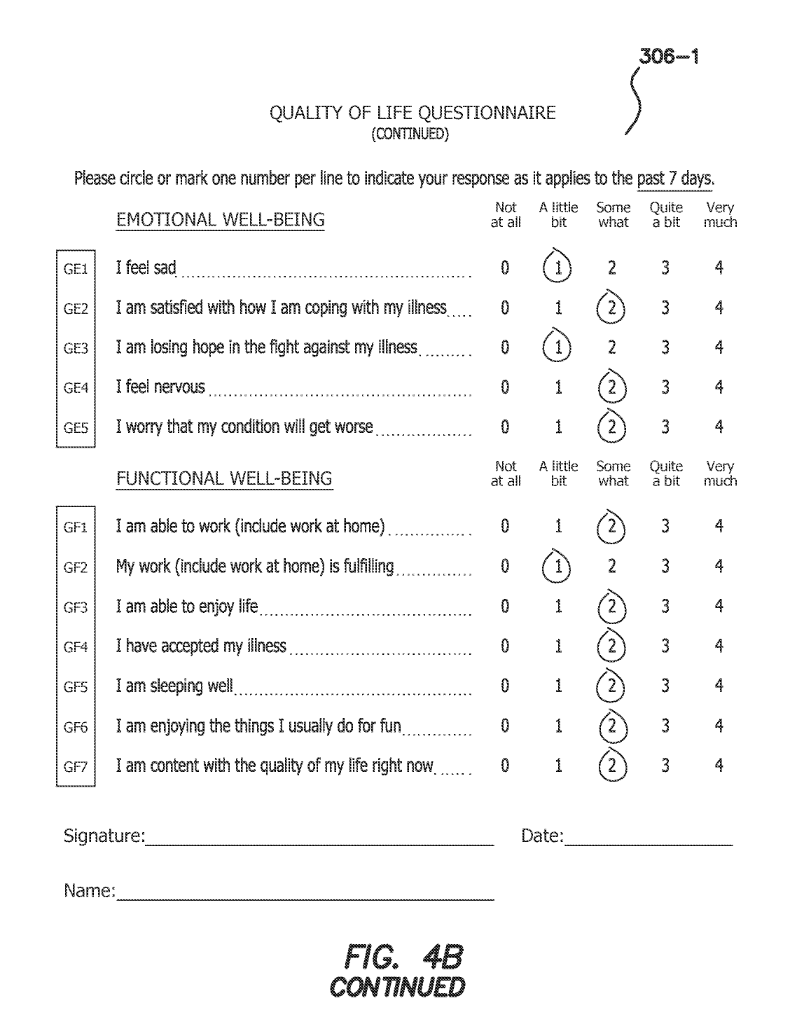

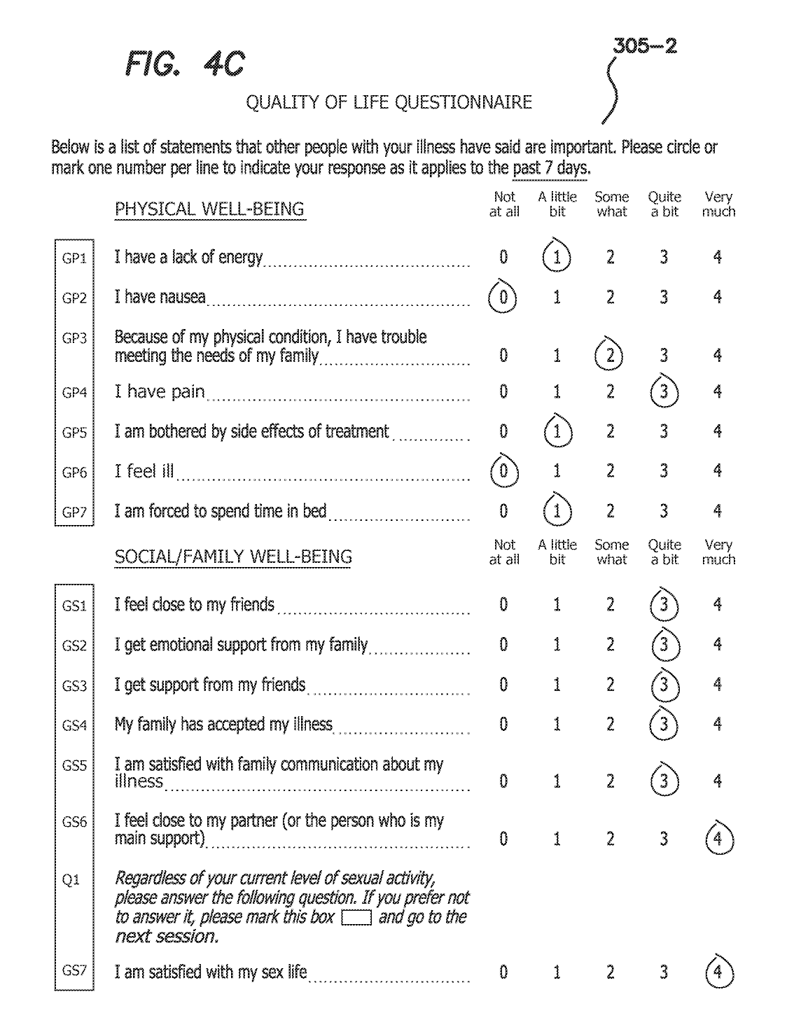

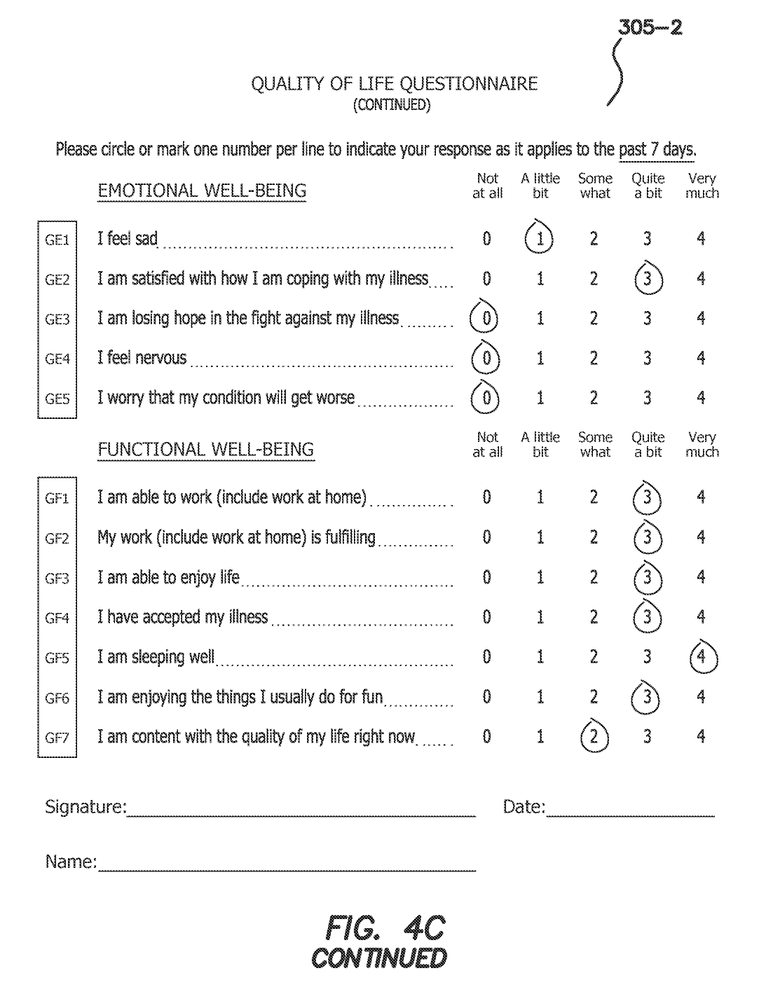

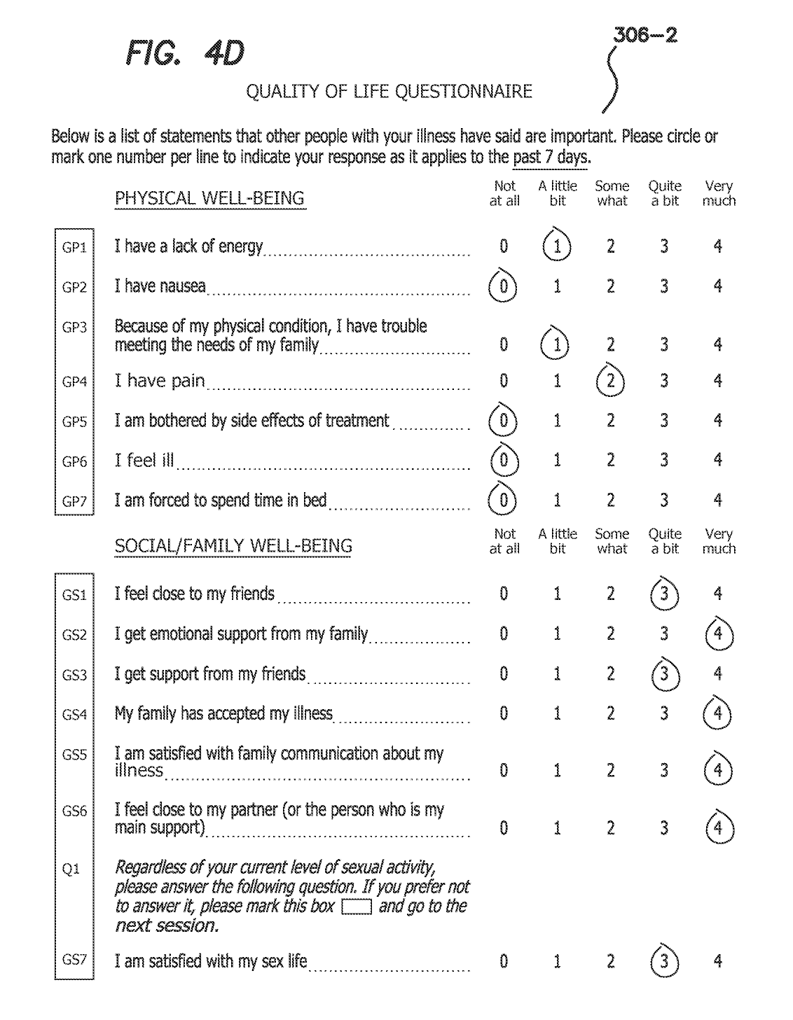

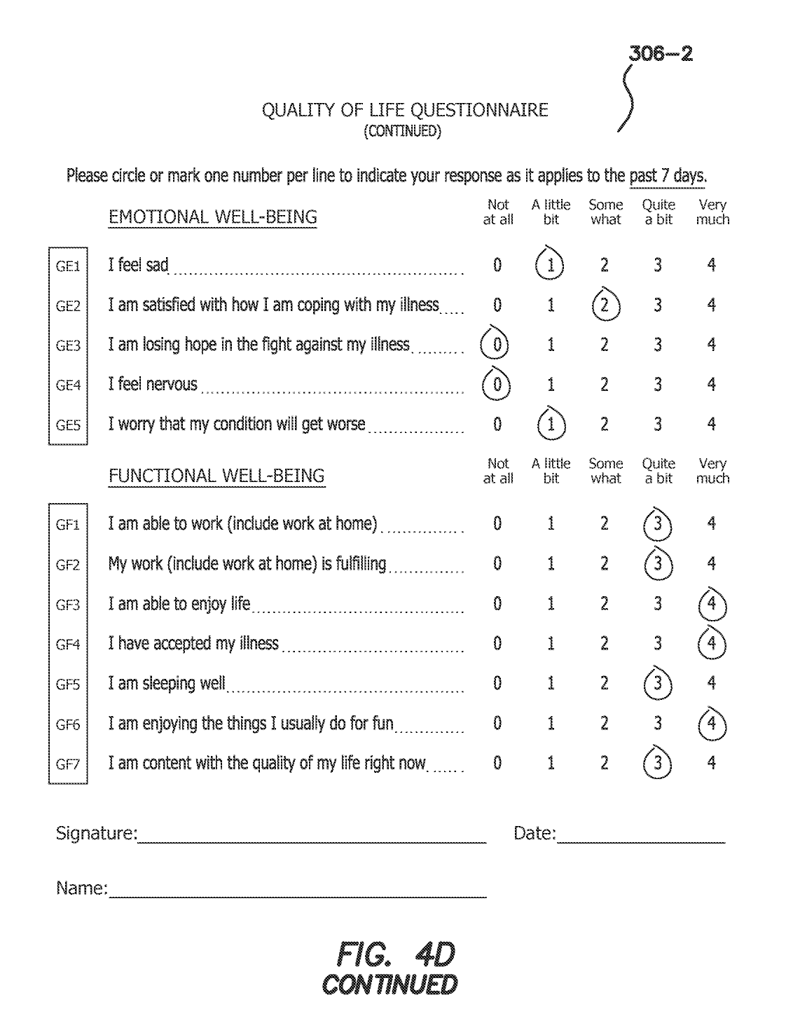

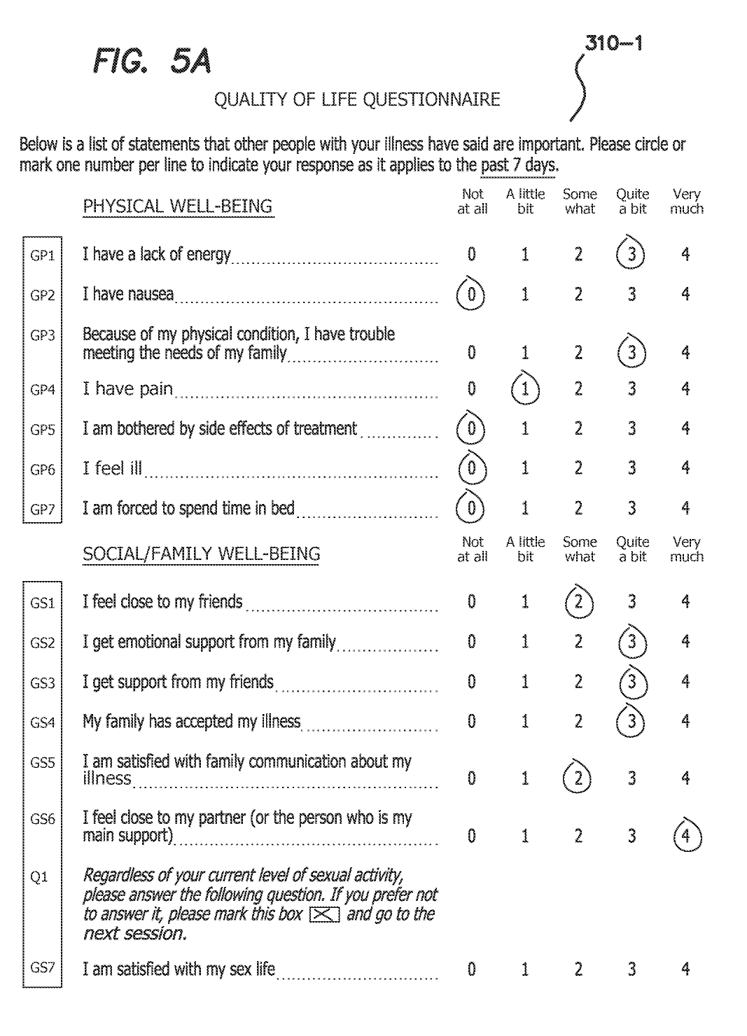

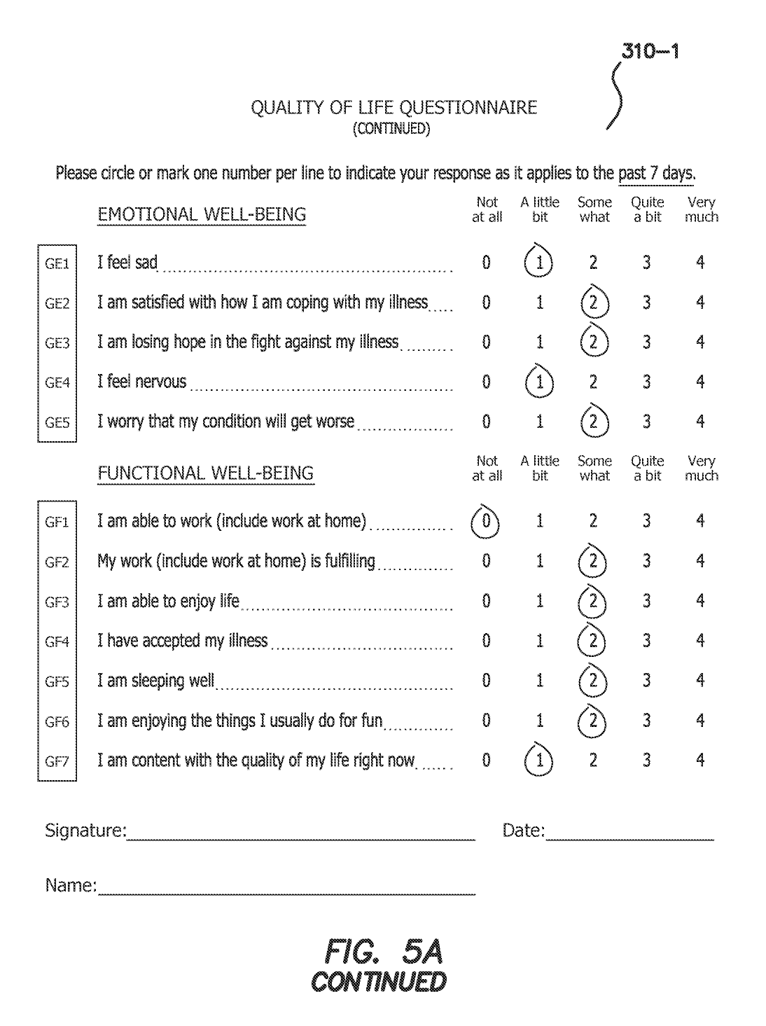

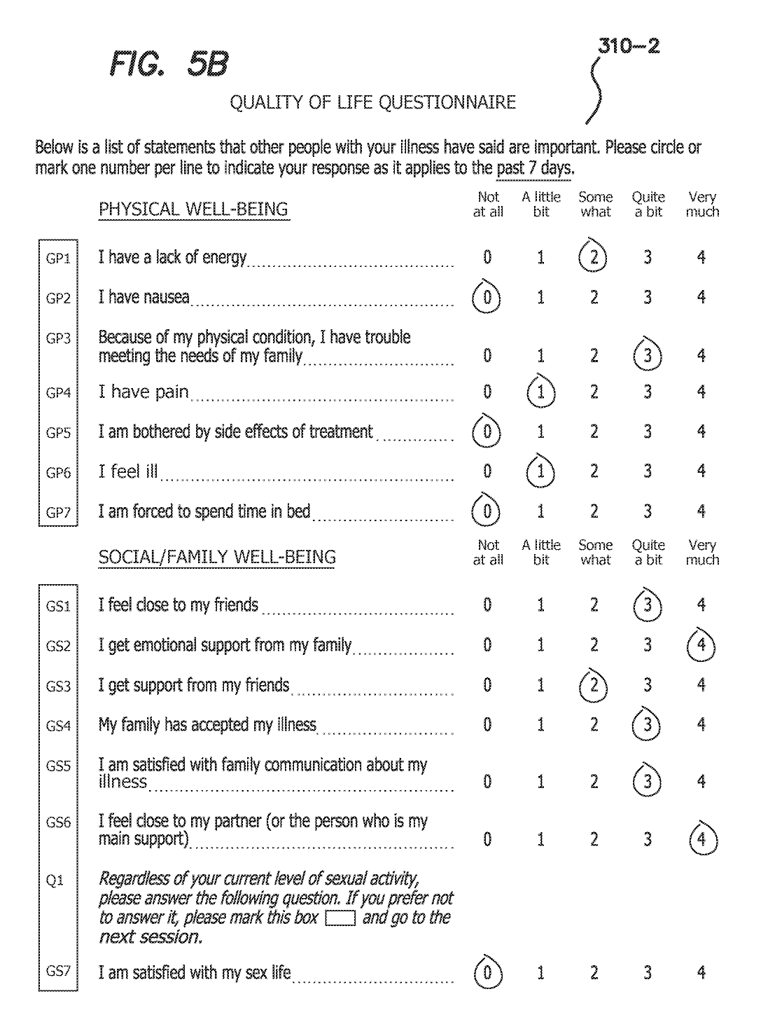

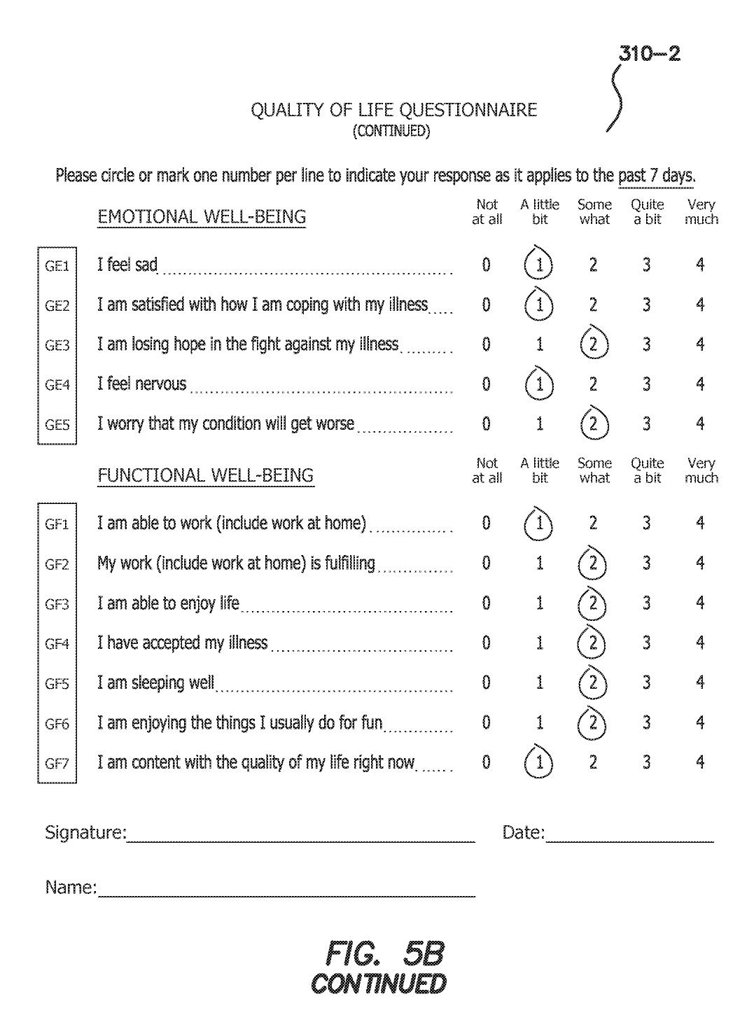

Numerous case studies on COPD patients were conducted using the intravenous injection and nebulizer infusion protocols. In general, the patients showed increased PO₂ readings; reduction in O₂ via nasal cannula; increased periods without need for O₂; and increased energy, stamina, activity and capacity for low action oxygen environment. As referenced below, other disesases were treated as well. FIGS. 3a-3l illustrate pre-treatment patient questionnaires 300-1 though 300-6 and corresponding post-treatment questionnaires 301-1 through 301-6 of Parkinson's patients being treated according to the embodiments of the present invention. FIGS. 4a-4d illustrate pre-treatment patient questionnaires 305-1 and 305-2 and post-treatment questionnaires 306-1 and 306-2 of COPD patients according to the embodiments of the present invention and FIGS. 5a-5b illustrate a pre-treatment patient questionnaire 310-1 and post-treatment questionnaire 310-2 of a MS patient according to the embodiments of the present invention.

As described herein, the embodiments of the present invention are directed to nutraceutical or pharmaceutical, such as a plant-based cyanobacteria phytochemical, Epogen, Neupogen or an adaptogen, for use in increasing a pluripotent stem cell count in mammals. In one embodiment, Table 1 lists an ingestion protocol for the nutraceutical or pharmaceutical. The increased stem cells may then be harvested, processed and returned to the patient for the treatment of various diseases as described herein.

Although the invention has been described in detail with reference to several embodiments, additional variations and modifications exist within the scope and spirit of the invention as described and defined in the following claims.

We claim:

1. An ex vivo pluripotent stem cell population comprising:
in vivo pluripotent stem cells increased in a mammal by delivering to a mammal a composition which increases in vivo pluripotent stem cells in the mammal, said in vivo pluripotent stem cells removed from the mammal to generate an ex vivo pluripotent stem cell population.

2. An ex vivo pluripotent stem cell population comprising:
in vivo pluripotent stem cells increased in a mammal by delivering to a mammal a composition which increases in vivo pluripotent stem cells in the mammal, said in vivo pluripotent stem cells removed from the mammal to generate said ex vivo pluripotent stem cell population, said ex vivo pluripotent stem cell population formulated to be infused back into the mammal to treat disease.

3. An ex vivo pluripotent stem cell population comprising:
in vivo pluripotent stem cells increased in a mammal by delivering to a mammal a composition which increases in vivo pluripotent stem cells in the mammal, said in vivo pluripotent stem cells removed from the mammal to generate said ex vivo pluripotent stem cell population, said ex vivo pluripotent stem cell population formulated to be infused back into the mammal to treat disease by:
(a) Nebulization;
(b) Intravenous bolus;
(c) Intranasal inhalation;
(d) Intra-spinal injection;
(e) Intra-articular injection;
(f) Topical cream; or
(g) Eye drops.
